SEQUENCE LISTING (139 sequences)

SEQ ID NO: 1 - DNA, 33 bp, Artificial sequence
  Synthetic antibody
caaagcctcgtacacagtgatggaaacacctac

SEQ ID NO: 2 - DNA, 9 bp, Artificial sequence
  Synthetic antibody
aaggtttct

SEQ ID NO: 3 - DNA, 27 bp, Artificial sequence
  Synthetic antibody
atgcaaggtgcacactggcctccgact

SEQ ID NO: 4 - DNA, 18 bp, Artificial sequence
  Synthetic antibody
cagagcattggcagctat

SEQ ID NO: 5 - DNA, 9 bp, Artificial sequence
  Synthetic antibody
tctgcatcc

SEQ ID NO: 6 - DNA, 27 bp, Artificial sequence
  Synthetic antibody
caacagagttacagtggcccgtacact

SEQ ID NO: 7 - DNA, 18 bp, Artificial sequence
  Synthetic antibody
cagggcattgccagtgct

SEQ ID NO: 8 - DNA, 9 bp, Artificial sequence
  Synthetic antibody
gatgcctcc

SEQ ID NO: 9 - DNA, 27 bp, Artificial sequence
  Synthetic antibody
caacaggctcacagtttcccgctcact

SEQ ID NO: 10 - DNA, 27 bp, Artificial sequence
  Synthetic antibody
agcagtgacgttggtgcatataactat

SEQ ID NO: 11 - DNA, 9 bp, Artificial sequence
  Synthetic antibody
gatgtcagt

SEQ ID NO: 12 - DNA, 30 bp, Artificial sequence
  Synthetic antibody
agctcatatacaaggagcaccactgtggta

SEQ ID NO: 13 - DNA, 27 bp, Artificial sequence
  Synthetic antibody
agcagtgacgttggtggttataactat

SEQ ID NO: 14 - DNA, 9 bp, Artificial sequence
  Synthetic antibody
gaggtcagt

SEQ ID NO: 15 - DNA, 30 bp, Artificial sequence
  Synthetic antibody
agctcatatgcaggcagcaacaatttggta

SEQ ID NO: 16 - DNA, 18 bp, Artificial sequence
  Synthetic antibody
caggacatcagaaatgat

SEQ ID NO: 17 - DNA, 9 bp, Artificial sequence
  Synthetic antibody
acagcaagt

SEQ ID NO: 18 - DNA, 27 bp, Artificial sequence
  Synthetic antibody
ctacaagattacaagtatcctctcact

SEQ ID NO: 19 - DNA, 27 bp, Artificial sequence
  Synthetic antibody
agcagtgacgttggtggttataactat

SEQ ID NO: 20 - DNA, 9 bp, Artificial sequence
  Synthetic antibody
gatgtcagt

SEQ ID NO: 21 - DNA, 33 bp, Artificial sequence
  Synthetic antibody
agctcatatacaagcagcagcactctcgtggta

SEQ ID NO: 22 - DNA, 24 bp, Artificial sequence
  Synthetic antibody
ggattcaccttcagtagctatgct

SEQ ID NO: 23 - DNA, 24 bp, Artificial sequence
  Synthetic antibody
atatcatatgatggaagtaataaa

SEQ ID NO: 24 - DNA, 48 bp, Artificial sequence
  Synthetic antibody
gcgagattgtcgaggttcggggatgaaagcctaaaagaccctgactac

SEQ ID NO: 25 - DNA, 24 bp, Artificial sequence
  Synthetic antibody
ggattcacctttgatgattatggc

SEQ ID NO: 26 - DNA, 24 bp, Artificial sequence
  Synthetic antibody
attaattggaatggtggtagcaca

SEQ ID NO: 27 - DNA, 42 bp, Artificial sequence
  Synthetic antibody
gcgagagcctctatggttcggggagtgtatgcttttgatatc

SEQ ID NO: 28 - DNA, 24 bp, Artificial sequence
  Synthetic antibody
ggaggcaccttcagcacctatgct

SEQ ID NO: 29 - DNA, 24 bp, Artificial sequence
  Synthetic antibody
atcatccctatctttggtacaaca

SEQ ID NO: 30 - DNA, 42 bp, Artificial sequence
  Synthetic antibody
gcgagagtttctgagctcggtgactactacgctatggacgtc

SEQ ID NO: 31 - DNA, 24 bp, Artificial sequence
  Synthetic antibody
ggttacacctttaccagctatggt

SEQ ID NO: 32 - DNA, 24 bp, Artificial sequence
  Synthetic antibody
atcagcgcttacaatggtaacaca

SEQ ID NO: 33 - DNA, 33 bp, Artificial sequence
  Synthetic antibody
gcgagagcaacaaggggaaatgcttttgatatc

SEQ ID NO: 34 - DNA, 24 bp, Artificial sequence
  Synthetic antibody
ggttacacctttaccagctacggt

SEQ ID NO: 35 - DNA, 24 bp, Artificial sequence
  Synthetic antibody
atcagcgcttacaatggtaacaca

SEQ ID NO: 36 - DNA, 33 bp, Artificial sequence
  Synthetic antibody
gcgagagccgtagggcagctggcctttgactac

SEQ ID NO: 37 - DNA, 24 bp, Artificial sequence
  Synthetic antibody
ggctacacctttaacagagatgct

SEQ ID NO: 38 - DNA, 24 bp, Artificial sequence
  Synthetic antibody
atcagcggttacaatggtaacaca

SEQ ID NO: 39 - DNA, 39 bp, Artificial sequence
  Synthetic antibody
gcgagagtcggcctacagccggatacaactatggaccac

SEQ ID NO: 40 - DNA, 24 bp, Artificial sequence
  Synthetic antibody
ggttacacctttaccagctacggt

SEQ ID NO: 41 - DNA, 24 bp, Artificial sequence
  Synthetic antibody
atcagcgcttacaatggtaacaca

SEQ ID NO: 42 - DNA, 33 bp, Artificial sequence
  Synthetic antibody
gcgagagccacgttcgggaatgcttttgatatc

SEQ ID NO: 43 - protein, 11 aa, Artificial sequence
  Synthetic antibody
QSLVHSDGNTY

SEQ ID NO: 44 - protein, 3 aa, Artificial sequence
  Synthetic antibody
KVS

SEQ ID NO: 45 - protein, 9 aa, Artificial sequence
  Synthetic antibody
MQGAHWPPT

SEQ ID NO: 46 - protein, 6 aa, Artificial sequence
  Synthetic antibody
QSIGSY

SEQ ID NO: 47 - protein, 3 aa, Artificial sequence
  Synthetic antibody
SAS

SEQ ID NO: 48 - protein, 9 aa, Artificial sequence
  Synthetic antibody
QQSYSGPYT

SEQ ID NO: 49 - protein, 6 aa, Artificial sequence
  Synthetic antibody
QGIASA

SEQ ID NO: 50 - protein, 3 aa, Artificial sequence
  Synthetic antibody
DAS

SEQ ID NO: 51 - protein, 9 aa, Artificial sequence
  Synthetic antibody
QQAHSFPLT

SEQ ID NO: 52 - protein, 9 aa, Artificial sequence
  Synthetic antibody
SSDVGAYNY

SEQ ID NO: 53 - protein, 3 aa, Artificial sequence
  Synthetic antibody
DVS

SEQ ID NO: 54 - protein, 10 aa, Artificial sequence
  Synthetic antibody
SSYTRSTTVV

SEQ ID NO: 55 - protein, 9 aa, Artificial sequence
  Synthetic antibody
SSDVGGYNY

SEQ ID NO: 56 - protein, 3 aa, Artificial sequence
  Synthetic antibody
EVS

SEQ ID NO: 57 - protein, 10 aa, Artificial sequence
  Synthetic antibody
SSYAGSNNLV

SEQ ID NO: 58 - protein, 6 aa, Artificial sequence
  Synthetic antibody
QDIRND

SEQ ID NO: 59 - protein, 3 aa, Artificial sequence
  Synthetic antibody
TAS

SEQ ID NO: 60 - protein, 9 aa, Artificial sequence
  Synthetic antibody
LQDYKYPLT

SEQ ID NO: 61 - protein, 9 aa, Artificial sequence
  Synthetic antibody
SSDVGGYNY

SEQ ID NO: 62 - protein, 3 aa, Artificial sequence
  Synthetic antibody
DVS

SEQ ID NO: 63 - protein, 11 aa, Artificial sequence
  Synthetic antibody
SSYTSSSTLVV

SEQ ID NO: 64 - protein, 8 aa, Artificial sequence
  Synthetic antibody
GFTFSSYA

SEQ ID NO: 65 - protein, 8 aa, Artificial sequence
  Synthetic antibody
ISYDGSNK

SEQ ID NO: 66 - protein, 16 aa, Artificial sequence
  Synthetic antibody
ARLSRFGDESLKDPDY

SEQ ID NO: 67 - protein, 8 aa, Artificial sequence
  Synthetic antibody
GFTFDDYG

SEQ ID NO: 68 - protein, 8 aa, Artificial sequence
  Synthetic antibody
INWNGGST

SEQ ID NO: 69 - protein, 14 aa, Artificial sequence
  Synthetic antibody
ARASMVRGVYAFDI

SEQ ID NO: 70 - protein, 8 aa, Artificial sequence
  Synthetic antibody
GGTFSTYA

SEQ ID NO: 71 - protein, 8 aa, Artificial sequence
  Synthetic antibody
IIPIFGTT

SEQ ID NO: 72 - protein, 14 aa, Artificial sequence
  Synthetic antibody
ARVSELGDYYAMDV

SEQ ID NO: 73 - protein, 8 aa, Artificial sequence
  Synthetic antibody
GYTFTSYG

SEQ ID NO: 74 - protein, 8 aa, Artificial sequence
  Synthetic antibody
ISAYNGNT

SEQ ID NO: 75 - protein, 11 aa, Artificial sequence
  Synthetic antibody
ARATRGNAFDI

SEQ ID NO: 76 - protein, 8 aa, Artificial sequence
  Synthetic antibody
GYTFTSYG

SEQ ID NO: 77 - protein, 8 aa, Artificial sequence
  Synthetic antibody
ISAYNGNT

SEQ ID NO: 78 - protein, 11 aa, Artificial sequence
  Synthetic antibody
ARAVGQLAFDY

SEQ ID NO: 79 - protein, 8 aa, Artificial sequence
  Synthetic antibody
GYTFNRDA

SEQ ID NO: 80 - protein, 8 aa, Artificial sequence
  Synthetic antibody
ISGYNGNT

SEQ ID NO: 81 - protein, 13 aa, Artificial sequence
  Synthetic antibody
ARVGLQPDTTMDH

SEQ ID NO: 82 - protein, 8 aa, Artificial sequence
  Synthetic antibody
GYTFTSYG

SEQ ID NO: 83 - protein, 8 aa, Artificial sequence
  Synthetic antibody
ISAYNGNT

SEQ ID NO: 84 - protein, 11 aa, Artificial sequence
  Synthetic antibody
ARATFGNAFDI

SEQ ID NO: 85-88 - skipped (4 entries)

SEQ ID NO: 89 - DNA, 369 bp, Artificial sequence
  Synthetic antibody
caggtgcagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagattgtcgaggttcggggatgaaagcctaaaagaccctgactactggggccagggaaccctggtcaccgtctcctca

SEQ ID NO: 90 - DNA, 363 bp, Artificial sequence
  Synthetic antibody
gaagtgcagctgttgcagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtattactgtgcgagagcctctatggttcggggagtgtatgcttttgatatctggggccaagggacaatggtcatcgtctcctca

SEQ ID NO: 91 - DNA, 363 bp, Artificial sequence
  Synthetic antibody
gaggtgcagctggtgcaatctgggggtgaggtgaagaagcctgggtcgtcggtgaaggtctcctgcaaggcttctggaggcaccttcagcacctatgctatcagctgggtgcgacaggcccctggacaagggcttgagtggatgggagggatcatccctatctttggtacaacaaattacgcacagaagtttcagggcagagtcacgattaccgcggacagatccacgagcacagcctacatggagttgagcagccttagatctgaagacacggccgtgtattactgtgcgagagtttctgagctcggtgactactacgctatggacgtctggggccaagggaccacggtcaccgtctcctca

SEQ ID NO: 92 - DNA, 354 bp, Artificial sequence
  Synthetic antibody
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagcaacaaggggaaatgcttttgatatctggggccaagggacaatggtcagcgtctcctca

SEQ ID NO: 93 - DNA, 354 bp, Artificial sequence
  Synthetic antibody
caggtgcagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagccgtagggcagctggcctttgactactggggccagggaaccctggtcaccgtctcctca

SEQ ID NO: 94 - DNA, 360 bp, Artificial sequence
  Synthetic antibody
gaagtgcagctggtgcagtctggggccgaggtgaagaagcctggggcctcagtgaaggtctcctgcaaggcttccggctacacctttaacagagatgctatcagctgggtgcgacaggcccctggacaagggcttgagtggatgggatggatcagcggttacaatggtaacacatactatgcacacaaggtcctgggcagagccaccatgaccacagacacatccacgaacacagcctacatggagctgaggagcctgacatctgacgacacggccatgtactactgtgcgagagtcggcctacagccggatacaactatggaccactggggccagggaaccctggtcaccgtctcctca

SEQ ID NO: 95 - DNA, 354 bp, Artificial sequence
  Synthetic antibody
caggtgcagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagccacgttcgggaatgcttttgatatctggggccaagggaccacggtcaccgtctcctca

SEQ ID NO: 96 - DNA, 339 bp, Artificial sequence
  Synthetic antibody
gatattgtgatgactcagtctccactctccctgcccgtcacccttggacagccggcctccatctcctgcaggtctagtcaaagcctcgtacacagtgatggaaacacctacttgaattggtttcagcagaggccaggccagtctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtgcacactggcctccgactttcggccctgggaccaagctggagatcaaacgt

SEQ ID NO: 97 - DNA, 321 bp, Artificial sequence
  Synthetic antibody
gacgtccagatgacccagtctccatcctccctgtctgcatctataggagacagagtcaccatcacttgccgggcgagtcagagcattggcagctatttaaattggtatcagcagagaccagggaaagcccctaaactcctcatctattctgcatccagtttgcagagtggggtcccatcaaggttcagtggcagtggatctcggtcagatttcactctcactatcgccagtctgcaacctgaagattttgcaacttactactgtcaacagagttacagtggcccgtacacttttggccaggggactaaagtggatatcaaa

SEQ ID NO: 98 - DNA, 321 bp, Artificial sequence
  Synthetic antibody
gacatccagatgacccagtctccctcctccctgtctgcatctgtaggagacagagtcaccattacctgtcgggcaagtcagggcattgccagtgctttagcctggtatcagcagaaaccagggaaagctcctaaactcctgatctatgatgcctccaatttggagagtggggtcccatcaaggttcagcggcagtggatctgggacagatttcactctcaccatcagcagcctgcagcctgaggattttgcaacttactattgtcaacaggctcacagtttcccgctcactttcggcggagggaccaagctggagatcaaa

SEQ ID NO: 99 - DNA, 330 bp, Artificial sequence
  Synthetic antibody
cagtctgccctgactcagcctgcctccgtgtctgggtctcctggacagtcgatcaccatctcctgcactggaaccagcagtgacgttggtgcatataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaaggagcaccactgtggtattcggcggagggaccaaggtcaccgtccta

SEQ ID NO: 100 - DNA, 330 bp, Artificial sequence
  Synthetic antibody
cagtctgccttgaatcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccgtctctgggctccaggctgaggatgaggctgattattactgcagctcatatgcaggcagcaacaatttggtattcggcggagggaccaaggtcaccgtccta

SEQ ID NO: 101 - DNA, 321 bp, Artificial sequence
  Synthetic antibody
gacatccagatgacccagtctccatcctccctgtctgcatctgtgggagacagagtcaccatcacttgccgggcaagtcaggacatcagaaatgatttagcctggtatcagcagaaaccagggaaagcccctaagctcctgatctatacagcaagtagtttacagactggggtcccatcgaggttcagcggcagtggatctggcacagatttcactctctccatcagcagcctgcagcctgaagattttgcaacttattactgtctacaagattacaagtatcctctcactttcggcggagggaccaagctggagatcaaa

SEQ ID NO: 102 - DNA, 333 bp, Artificial sequence
  Synthetic antibody
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactctcgtggtattcggcggagggaccaaggtcaccgtccta

SEQ ID NO: 103 - protein, 123 aa, Artificial sequence
  Synthetic antibody
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARLSRFGDESLKDPDYWGQGTLVTVSS

SEQ ID NO: 104 - protein, 121 aa, Artificial sequence
  Synthetic antibody
EVQLLQSGGGVVRPGGSLRLSCAASGFTFDDYGMSWVRQAPGKGLEWVSGINWNGGSTGYADSVKGRFTISRDNAKNSLYLQMNSLRAEDTALYYCARASMVRGVYAFDIWGQGTMVIVSS

SEQ ID NO: 105 - protein, 121 aa, Artificial sequence
  Synthetic antibody
EVQLVQSGGEVKKPGSSVKVSCKASGGTFSTYAISWVRQAPGQGLEWMGGIIPIFGTTNYAQKFQGRVTITADRSTSTAYMELSSLRSEDTAVYYCARVSELGDYYAMDVWGQGTTVTVSS

SEQ ID NO: 106 - protein, 118 aa, Artificial sequence
  Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARATRGNAFDIWGQGTMVSVSS

SEQ ID NO: 107 - protein, 118 aa, Artificial sequence
  Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARAVGQLAFDYWGQGTLVTVSS

SEQ ID NO: 108 - protein, 120 aa, Artificial sequence
  Synthetic antibody
EVQLVQSGAEVKKPGASVKVSCKASGYTFNRDAISWVRQAPGQGLEWMGWISGYNGNTYYAHKVLGRATMTTDTSTNTAYMELRSLTSDDTAMYYCARVGLQPDTTMDHWGQGTLVTVSS

SEQ ID NO: 109 - protein, 118 aa, Artificial sequence
  Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARATFGNAFDIWGQGTTVTVSS

SEQ ID NO: 110 - protein, 112 aa, Artificial sequence
  Synthetic antibody
DIVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGAHWPPTFGPGTKLEIK

SEQ ID NO: 111 - protein, 107 aa, Artificial sequence
  Synthetic antibody
DVQMTQSPSSLSASIGDRVTITCRASQSIGSYLNWYQQRPGKAPKLLIYSASSLQSGVPSRFSGSGSRSDFTLTIASLQPEDFATYYCQQSYSGPYTFGQGTKVDIK

SEQ ID NO: 112 - protein, 107 aa, Artificial sequence
  Synthetic antibody
DIQMTQSPSSLSASVGDRVTITCRASQGIASALAWYQQKPGKAPKLLIYDASNLESGVPSRFSGSGSGTDFTLTISSLQPEDFATYYCQQAHSFPLTFGGGTKLEIK

SEQ ID NO: 113 - protein, 110 aa, Artificial sequence
  Synthetic antibody
QSALTQPASVSGSPGQSITISCTGTSSDVGAYNYVSWYQQHPGKAPKLMIYDVSNRPSGVSNRFSGSKSGNTASLTISGLQAEDEADYYCSSYTRSTTVVFGGGTKVTVL

SEQ ID NO: 114 - protein, 110 aa, Artificial sequence
  Synthetic antibody
QSALNQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKVTVL

SEQ ID NO: 115 - protein, 107 aa, Artificial sequence
  Synthetic antibody
DIQMTQSPSSLSASVGDRVTITCRASQDIRNDLAWYQQKPGKAPKLLIYTASSLQTGVPSRFSGSGSGTDFTLSISSLQPEDFATYYCLQDYKYPLTFGGGTKLEIK

SEQ ID NO: 116 - protein, 111 aa, Artificial sequence
  Synthetic antibody
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVVFGGGTKVTVL

SEQ ID NO: 117 - DNA, 20 bp, Artificial sequence
  Synthetic antibody
aactcattcccttggtggtg

SEQ ID NO: 118 - DNA, 20 bp, Artificial sequence
  Synthetic antibody
gtagggtccacagtttccca

SEQ ID NO: 119 - DNA, 20 bp, Artificial sequence
  Synthetic antibody
ggaaaagaaacccagagcct

SEQ ID NO: 120 - DNA, 20 bp, Artificial sequence
  Synthetic antibody
ggactttttgcagctcctca

SEQ ID NO: 121 - DNA, 17 bp, Artificial sequence
  Synthetic antibody
tgtggggaaggggctgt

SEQ ID NO: 122 - DNA, 23 bp, Artificial sequence
  Synthetic antibody
ccaccaaacaagttagagaatgc

SEQ ID NO: 123 - DNA, 20 bp, Artificial sequence
  Synthetic antibody
gtgatttgcttttgcctcct

SEQ ID NO: 124 - DNA, 20 bp, Artificial sequence
  Synthetic antibody
gccacagcatctgggtattt

SEQ ID NO: 125 - DNA, 21 bp, Artificial sequence
  Synthetic antibody
cccttctgcctgacacctttc

SEQ ID NO: 126 - DNA, 19 bp, Artificial sequence
  Synthetic antibody
gcaaccctgccagcaattt

SEQ ID NO: 127 - DNA, 18 bp, Artificial sequence
  Synthetic antibody
cgctacctggccatcgtc

SEQ ID NO: 128 - DNA, 23 bp, Artificial sequence
  Synthetic antibody
catagaccaccttttcagccaac

SEQ ID NO: 129 - protein, 83 aa, Homo sapiens
MSFFQLLMKRKELIPLVVFMTVAAGGASSFAVYSLWKTDVILDRKKNPEPWETVDPTVPQKLITINQQWKPIEELQNVQRVTK

SEQ ID NO: 130 - protein, 83 aa, Pan troglodytes; identical to SEQ ID NO: 129

SEQ ID NO: 131 - protein, 83 aa, Pan paniscus; identical to SEQ ID NO: 129

SEQ ID NO: 132 - protein, 83 aa, Pongo abelii [sequence omitted]

SEQ ID NO: 133 - protein, 83 aa, Gorilla gorilla; identical to SEQ ID NO: 129

SEQ ID NO: 134 - protein, 83 aa, Macaca fascicularis
MSFFQLLMKRKELIPLVLFVSVAAGGASSFAVYSLWKTDVILDRKKNPEPWETVDPTIPQKLITINQQWKPIEELQNVQRVTK

SEQ ID NO: 135 - protein, 83 aa, Macaca mulatta
MSFFQLLMKRKELIPLVLFVSVAAGGASSFAVYSLWKTDVILDRKKNPEPWETVDPTIPQKLITINQQWKPIEELQNVRRVTK

SEQ ID NO: 136 - protein, 83 aa, Mus musculus
MGVFQILMKNKELIPLAFFISVAATGATSFALYALKKTDVVIDRKRNPEPWEMVDPTQPQKLITINQQWKPVEELQKVRRATR

SEQ ID NO: 137 - protein, 83 aa, Heterocephalus glaber
MGVGQTLMKKKELIPLAVIMTVAAGGALSFAVYSLRKTDVIFDRKGNPEPWETVDPSVPQKLITINQQWKPIEELQKVRGATK

SEQ ID NO: 138 - protein, 83 aa, Otolemur garnettii
MGVFQLLMKKKELIPLAVFITFAAVGASSFAVYSLGKSDVIIDRKRNPEPWENVDPNVPQKLVTINQQWKPIEELQKIRRATK

SEQ ID NO: 139 - protein, 83 aa, Pteropus vampyrus
MNLFQLLMKKKELIPLAVIMTAAACGALSFALYSLKKTDVIIDRKRNPEPWENVDPNVPRKLITINQQWKPIEELQKARRASR

(19) United States
(12) Patent Application Publication
(10) Pub. No.: US 2021/0371508 A1
(43) Pub. Date: Dec. 2, 2021

(54) NMES1 ANTIBODIES AND METHODS OF USE THEREOF

(71) Applicant: The Board of Regents of the University of Texas System, Austin, TX (US)

(72) Inventors: Ningyan ZHANG, Houston, TX (US); Zhiqiang AN, Houston, TX (US); Zhiqiang KU, Houston, TX (US); Yihong WAN, Dallas, TX (US); Jing KRZESZINSKI, Dallas, TX (US)

(73) Assignee: The Board of Regents of the University of Texas System, Austin, TX (US)

(21) Appl. No.: 17/332,790

(22) Filed: May 27, 2021

Related U.S. Application Data
(60) Provisional application No. 63/031,416, filed on May 28, 2020.

(51) Int. Cl. C07K 16/18; A61K 45/06; G01N 33/574; A61K 47/68

(52) U.S. Cl. CPC C07K 16/18; A61K 45/06; G01N 33/574; A61K 2039/505; C07K 2317/565; G01N 2333/47; A61K 47/6801

(57) ABSTRACT

Provided herein are methods for treating cancer, such as breast cancer, by administering an inhibitor of NMES1. Further provided herein are NMES1 monoclonal antibodies which may be used to detect or treat cancer.

### PRIORITY CLAIM

This application claims benefit of priority to U.S. Provisional Application Ser. No. 63/031,416, filed May 28, 2020, the entire contents of which is hereby incorporated by reference.

### INCORPORATION OF SEQUENCE LISTING

The sequence listing that is contained in the file named “UTSHP0367US_ST25.txt”, which is 50 KB (as measured in Microsoft Windows) and was created on May 27, 2021, is filed herewith by electronic submission and is incorporated by reference herein.

### BACKGROUND

### 1. Field

The present invention relates generally to the field of molecular biology. More particularly, it concerns NMES1 as a biomarker for cancer and methods of targeting NMES1 for the treatment of cancer, such as a monoclonal NMES1 antibody.

### 2. Description of Related Art

Breast cancer mortality is mostly connected with distant metastasis to other organs. Over 75% of breast cancer recurrences involve bone metastasis (Coleman, 2001). Once tumors metastasize to bone, the patients are usually incurable: only 20% of women with breast cancer are still alive 5 years after the discovery of bone metastasis. The consequences of bone metastases are devastating: severe pain, pathologic fractures, life-threatening hypercalcemia, anemia, spinal cord compression, limited mobility and eventually mortality. Current treatment strategies are limited to alleviating bone destructions with osteoclast inhibitor drugs when skeletal metastases are detected, however these drugs are only palliative without any increase in patient survival, leaving the clinical management of breast cancer patients and the improvement of their life expectancy an urgent yet daunting challenge. Hence, discovery of novel determinants for breast cancer malignancy is crucial for revealing not only new disease mechanisms but also innovative therapeutic strategies.

### SUMMARY

In certain embodiments, the present disclosure provides an isolated monoclonal antibody, wherein the antibody specifically binds secreted normal mucosa of esophagus specific 1 (NMES1), wherein the antibody comprises (a) V_{L} domain CDRs 1-3 (SEQ ID NOs: 43-45) and V_{H} domain CDRs 1-3 (SEQ ID NOs:63-66); (b) V_{L} domain CDRs 1-3 (SEQ ID NOs: 46-48) and V_{H} domain CDRs 1-3 (SEQ ID NOs: 67-69); (c) V_{L} domain CDRs 1-3 (SEQ ID NOs: 49-51) and V_{H} domain CDRs 1-3 (SEQ ID NOs:70-72); (d) V_{L} domain CDRs 1-3 (SEQ ID NOs: 52-54) and V_{H} domain CDRs 1-3 (SEQ ID NOs:73-75); (e) V_{L} domain CDRs 1-3 (SEQ ID NOs: 55-57) and V_{H} domain CDRs 1-3 (SEQ ID NOs:76-78); (f) V_{L} domain CDRs 1-3 (SEQ ID NOs: 58-60) and V_{H} domain CDRs 1-3 (SEQ ID NOs:79-81); or (g) V_{L} domain CDRs 1-3 (SEQ ID NOs: 61-63) and V_{H} domain CDRs 1-3 (SEQ ID NOs:82-84).

the antibody comprises V_{L} domain CDRs 1-3 (SEQ ID NOs: 43-45) and V_{H} domain CDRs 1-3 (SEQ ID NOs:63-66).

In some aspects, the antibody comprises a V_{H} domain at least about 80% (e.g., 85%, 90%, 91%, 92%, 93%, 94%, 95%, 96%, 97%, 98%, or 99%) identical to the V_{H} domain (SEQ ID NO: 103) of NMES-1 and a V_{L} domain (SEQ ID NO:110) at least about 80% (e.g., 85%, 90%, 91%, 92%, 93%, 94%, 95%, 96%, 97%, 98%, or 99%) identical to the V_{L} domain of NMES-1. In certain aspects, the antibody comprises a V_{H} domain identical to the V_{H} domain (SEQ ID NO: 103) of NMES-1 and a V_{L} domain (SEQ ID NO: 110) identical to the V_{L} domain NMES-1.

In certain aspects, the antibody comprises a V_{H} domain at least about 80% (e.g., 85%, 90%, 91%, 92%, 93%, 94%, 95%, 96%, 97%, 98%, or 99%) identical to the V_{H} domain (SEQ ID NOs:104-109) of NMES-3, NMES-4, NMES-5, NMES-6, NMES-7, or NMES-10 and a V_{L} domain (SEQ ID NOs: 111-116) at least about 80% (e.g., 85%, 90%, 91%, 92%, 93%, 94%, 95%, 96%, 97%, 98%, or 99%) identical to the V_{L} domain of NMES-3, NMES-4, NMES-5, NMES-6, NMES-7, or NMES-10. In certain aspects, the antibody comprises a V_{H} domain identical to the V_{H} domain (SEQ ID NO: 104-109) of NMES-3, NMES-4, NMES-5, NMES-6, NMES-7, or NMES-10 NMES-1 and a V_{L} domain (SEQ ID NO: 111-116) identical to the V_{L} domain NMES-3, NMES-4, NMES-5, NMES-6, NMES-7, or NMES-10.

In some aspects, the antibody is recombinant. In certain aspects, the antibody is an IgG, IgM, IgA or an antigen binding fragment thereof. In particular aspects, the antibody is a Fab′, a F(ab′)2, a F(ab′)3, a monovalent scFv, a bivalent scFv, or a single domain antibody. In some aspects, the antibody is a human, humanized antibody or de-immunized antibody. In certain aspects, the antibody is conjugated to an imaging agent, a chemotherapeutic agent, a toxin or a radionuclide.

A further embodiment provides a composition comprising an antibody of the present embodiments or aspects thereof in a pharmaceutically acceptable carrier. Also provided herein is an isolated polynucleotide molecule comprising a nucleic acid sequence encoding an antibody of the present embodiments or aspects thereof.

Another embodiment provides a recombinant polypeptide comprising an antibody V_{H} domain comprising: (a) V_{L} domain CDRs 1-3 (SEQ ID NOs: 43-45) and V_{H} domain CDRs 1-3 (SEQ ID NOs:63-66); (b) V_{L} domain CDRs 1-3 (SEQ ID NOs: 46-48) and V_{H} domain CDRs 1-3 (SEQ ID NOs: 67-69); (c) V_{L} domain CDRs 1-3 (SEQ ID NOs: 49-51) and V_{H} domain CDRs 1-3 (SEQ ID NOs:70-72); (d) V_{L} domain CDRs 1-3 (SEQ ID NOs: 52-54) and V_{H} domain CDRs 1-3 (SEQ ID NOs:73-75); (e) V_{L} domain CDRs 1-3 (SEQ ID NOs: 55-57) and V_{H} domain CDRs 1-3 (SEQ ID NOs:76-78); (f) V_{L} domain CDRs 1-3 (SEQ ID NOs: 58-60) and V_{H} domain CDRs 1-3 (SEQ ID NOs:79-81); or (g) V_{L} domain CDRs 1-3 (SEQ ID NOs: 61-63) and V_{H} domain CDRs 1-3 (SEQ ID NOs:82-84). In yet another embodiment, there is provided an isolated polynucleotide molecule comprising a nucleic acid sequence encoding a polypeptide of the present embodiments.

A further embodiment provides a host cell comprising one or more polynucleotide molecule(s) encoding an antibody of the present embodiments or aspects thereof or a recombinant polypeptide of the present embodiments. In some aspects, the host cell is a mammalian cell, a yeast cell, a bacterial cell, a ciliate cell or an insect cell.

Another embodiment provides a pharmaceutical composition comprising a NMES1 antibody of the present embodiments or aspects thereof and a pharmaceutical carrier.

A composition comprising an effective amount of a NMES1 antibody of any of the present embodiments or aspects thereof for the treatment of cancer in a subject.

Further provided herein is the use of a composition comprising an effective amount of a NMES1 antibody of the present embodiments or aspects thereof for the treatment of cancer in a subject.

Another embodiment provides a method for treating cancer in a subject comprising administering an effective amount of an inhibitor of NMES1 to the subject.

In some aspects, the NMES1 inhibitor is an antibody of the present embodiments or aspects thereof. In certain aspects, the NMES1 inhibitor is short interfering RNA (siRNA) or short hairpin (shRNA) to NMES1 or transferrin receptor 1 (TFRC). In some aspects, the NMES1 inhibitor is a small molecule inhibitor.

In certain aspects, the cancer is breast cancer or renal cell carcinoma. In particular aspects, the cancer is metastatic breast cancer. In some aspects, the cancer has metastasized to the bone, such as breast cancer bone metastasis.

In particular aspects, the NMES1 inhibitor results in decreased iron uptake by cancer cells and/or decreased expression of bone metastatic genes. In some aspects, the bone metastatic genes are COX2 or CXCR4. In specific aspects, the NMES1 inhibitor results in decreased bone lesion.

In some aspects, the NMES1 inhibitor is administered intravenously, intradermally, intratumorally, intramuscularly, intraperitoneally, subcutaneously, or locally. In particular aspects, the NMES1 inhibitor is administered intravenously.

In additional aspects, the method further comprises administering at least a second anticancer therapy to the subject. In some aspects, the second anticancer therapy is a surgical therapy, chemotherapy, radiation therapy, cryotherapy, hormonal therapy, immunotherapy or cytokine therapy.

A further embodiment provides a method for detecting a cancer in a subject comprising measuring elevated secreted NMES1 relative to a control in a sample from the subject. In some aspects, the testing comprises contacting the sample with an antibody of the present embodiments or aspects thereof. In certain aspects, measuring comprises performing qRT-PCR, microarray analysis, ELISA, IHC, or western blot analysis. In some aspects, the sample is a blood sample, such as a serum sample. In some aspects, the method if further defined as an in vitro method.

Another embodiment provides an in vitro method of detecting secreted NMES1 in a sample comprising detecting an elevated level of NMES1 by measuring binding of a NMES1 antibody of the present embodiments or aspects thereof. In some aspects, the sample is blood, a tissue biopsy, fine needle aspirate, saliva, or urine. In certain aspects, the sample is a serum sample. In some aspects, the elevated level of secreted NMES1 as compared to a control identifies the samples as a cancer sample. In certain aspects, the cancer is breast cancer, such as metastatic breast cancer. In specific aspects, the elevated level of NMES1 as compared to a control indicates a poor prognosis.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1T: NMES1 promotes breast cancer and bone metastasis. (FIG. 1A) RNA expression of NMES1 was higher in bone-metastatic 1833 subline vs. MDA-MB-231 parental human breast cancer cell line (left), and in bone-metastatic 4T1.2 subline vs. 4T1 parental mouse mammary tumor cell line (right). (FIG. 1B) Protein expression of NMES1 was higher in 1833 cells vs. 231 cells. (FIG. 1C) TCGA BRCA data analysis showed that compared with normal breast samples, breast cancer lesions displayed higher NMES1 expression. Normal Breast (n=111); Infiltrating Ductal Carcinoma (n=765); Infiltrating Lobular Carcinoma (n=172); Medullary Carcinoma (n=6); Mixed Histology (n=29); Mucinous Carcinoma (n=15); Other Histology (n=44). (FIG. 1D) Analysis of a microarray dataset showed that NMES1 expression was significantly higher in bone metastatic (n=6) vs. disseminated (n=8) breast cancer cells. (FIGS. 1E-1H) NMES1 overexpression in MDA-MB-231 cells resulted in higher NMES1 mRNA (FIG. 1E) and protein (FIG. 1F), leading to increased cancer cell proliferation (FIG. 1G) and migration (FIG. 111). BoM-1833 human breast cancer cells were quantified by luciferase readout (n=6). (FIG. 1I-1L) NMES1 knockdown in BoM-1833 cells resulted in lower NMES1 mRNA (FIG. H) and protein (FIG. 1J), leading to decreased cancer cell proliferation (FIG. 1K) and migration (FIG. 1L). (FIGS. 1M-1P) NMES1 overexpression in MDA-MB-231 cells increased bone metastases. (FIGS. 1Q-1T) NMES1 knockdown in BoM-1833 cells decreased bone metastases. (FIGS. 1M, 1Q) Quantification of KJ signals. (FIGS. 1N, 1R) Representative BLI images. (FIGS. 1O, 1S) X-ray images showing osteolytic lesions (avows). (FIGS. 1P, 1T) Quantification of the number of bone metastatic sites. *P<0.05, **P<0.01, ***P<0.001, ****P<0.0001; n.s., non-significant; error bars represent SD, except SEM for panel c, d, m, q, p, t.

FIGS. 2A-2H: NMES1 is a secreted protein. (FIG. 2A) Sequence alignment of NMES1 proteins from different species. (SEQ ID NOS: 129-139) (FIGS. 2B-2D) NMES1 protein was detected in cancer cell conditioned medium and quantified by western blot (FIG. 2B) and ELISA (FIGS. 2C-2D). (FIG. 2E) Higher amount of circulating NMES1 was detected in mice bearing bone metastasis induced by MDA-MB-231 xenograft. (n=5). (FIGS. 2F-2G) Recombinant human NMES1 (rhNMES1) stimulated 231 cancer cell proliferation (FIG. 2F) and migration (FIG. 2G) (n=4). (FIG. 2H) rhNMES1 treatment enhanced cancer cell expression of pro-metastatic genes (n=3). *P<0.05, **P<0.01, ***P<0.001, ****P<0.0001; n.s., non-significant; error bars represent SD, except SEM for panel f, g.

FIGS. 3A-3I: NMES1 stabilizes TFRC protein to enhance cancer cell iron uptake. (FIG. 3A) Cell compartment fractionation followed by western blot shows that NMES1 is localized exclusively in the membrane fraction. of 1833 cells. (FIG. 3B) Co-IP followed by western blot in control or NMES1-KD 1833 cells show that NMES1 interacts with TFRC, and NMES1 deletion reduces TFRC protein level. (FIG. 3C) Knockdown of NMES1 didn't affect TFRC mRNA level (n=3). (FIG. 3D) NMES1 enhanced TFRC stability. Cells were treated with 50 μg/ml CHX for indicated time before harvest for western blot. (FIG. 3E) Immunofluorescence staining shows that NMES1 co-localizes with a lysosomal marker Lamp1 in 1833 cells. (FIG. 3F) Chloroquine dose-dependently rescues the TFRC protein reduction in NMES1-KD 1833 cells shown by western blot, indicating that NMES1 attenuates TFRC lysosomal degradation. Cells were treated with chloroquine at the indicated concentration for 4 hours before harvest. (FIG. 3G) MG132 did not rescue the TFRC protein reduction in NMES1-KD 1833 cells shown by western blot, indicating that NMES1 does not mainly regulate TFRC protein stability via proteasome pathway. Cells were treated with or without 50 μM MG132 for 5 hours before harvest. (FIGS. 3H-31) Cancer cell iron import was reduced by NMES1 KD (FIG. 3H) but enhanced by NMES1 OE in a TFRC-dependent manner (FIG. 3I) (n=3). Iron uptake=Total Cellular Iron _{5 μg/ml Tf culture}−Total Cellular Iron _{0 μg/ml Tf culture}. *P<0.05, **P<0.01, ***P<0.001, ****P<0.0001; n.s., non-significant; error bars represent SD, except SEM for panel c, h, i.

FIGS. 4A-4K: TFRC mediates NMES1 regulation of tumor growth and bone metastasis. (FIGS. 4A-4B) TFRC KD alleviated the stimulation of proliferation by NMES1 overexpression in MDA-MB-231 cells (FIG. 4A) or by rhNMES1 treatment in BoM-1833 cells (FIG. 4B) (n=6). (FIG. 4C) TFRC KD abolished the effect of NMES1 KD on BoM-1833 cell proliferation as NMES1 deletion could not further decrease proliferation in NMES1/TFRC double KD cells compared to TFRC KD cells (n=6). (FIG. 4D) TFRC KD alleviated NMES1 stimulation of cancer cell migration (n=4). (FIG. 4E) Western blot confirmed protein reduction in KD cells. (FIGS. 4F-4G) Primary tumor growth was similarly reduced by NMES1 KD and TFRC KD, and absence of TFRC abolished the effect of NMES1 KD. BoM-1833 cells were injected into the mammary fat pad of 8-week-old female nude mice (n=8). Tumors were collected and weighed 5 weeks later. (FIG. 4F) Images of the tumors. NT, not detected. (FIG. 4G) Quantification of tumor weight. (FIG. 4H) Expression of pro-metastatic genes in NMES1 and/or TFRC KD cells (n=3). (FIGS. 4I-4K) Bone metastasis was similarly reduced by NMES1 KD and TFRC KD, and absence of TFRC abolished the effect of NMES1 KD. BoM-1833 cells were intracardiacally injected into 5-week-old female nude mice (n=10). (i) Representative BLI images. (FIG. 4J) Quantification of BLI signals. (FIG. 4K) Quantification of the number of bone metastatic sites. *P<0.05, **P<0.01, ***P<0.001, ****P<0.0001; n.s., non-significant; error bars represent SEM.

FIGS. 5A-5N: NMES1 blocking antibodies impede bone metastasis. (FIGS. 5A-5B) A polyclonal anti-NMES1 (α-N) antibody significantly decreased the proliferation (FIG. 5A) and migration (FIG. 5B) of 1833 cells but not NMES1-KD 1833 cells, indicating that the effects were specific for NMES1 blockade. (FIGS. 5C-5F) A polyclonal anti-NMES1 antibody dosage-dependently reduced bone metastasis (FIG. 5C) Serum NMES1 levels were decreased by anti-NMES1 treatment. (n=5). (FIG. 5D) Quantification of BLI signals. (n=5). (FIG. 5E) Representative BLI images and X-ray images. (FIG. 5F) Kaplan-Meier plot of time to metastasis. (FIG. 5G) A diagram of the procedure for the generation of neutralizing antibodies for human NMES1. (FIG. 5H) ELISA determination of EC50 of NMES1 antibodies (n=2). (FIG. 5I) Co-IP analysis showed that monoclonal antibody NM-1 reduced the interaction between NMES1 and TFRC. Lysate from 231 cells that over-express V5-hNMES1 were incubated with 1 μg of NM-1 or isotype control overnight, immunoprecipitated with anti-V5 or IgG control, and then subjected to western blotting for the indicated proteins. (FIGS. 5J-5K) Humanized monoclonal anti-NMES1 antibodies (NM-1, NM-3, NM-4, NM-7) significantly decreased the proliferation (FIG. 5J) and migration (FIG. 5K) of 1833 cells (left) but not NMES1-KD 1833 cells (right). The polyclonal anti-NMES1 served as a positive control. Negative controls were corresponding isotype IgG. (FIGS. 5L-5N) A humanized monoclonal anti-NMES1 antibody (NM-1) significantly reduced bone metastases (n=4-5). (FIG. 5L) Quantification of BLI signals. (FIG. 5M) Quantification of the number of bone metastatic sites. (FIG. 5N) Kaplan-Meier plot of time to metastasis. *P<0.05, **P<0.01, ***P<0.001, ****P<0.0001; n.s., non-significant; error bars represent SD, except SEM for panel a, b, e, j-m.

FIGS. 6A-6B: Growth of 4T1.2 cells was increased by NMES1 overexpression and decreased by NMES1 knockdown. (FIG. 6A) Western blot showing NMES1 protein level in NMES1-OE and NMES1-KD cells. (FIG. 6B) Quantification of cell growth using luciferase readout (n=6). **P<0.01, ****P<0.0001; error bars indicate SD.

FIGS. 7A-7B: Effects of mAb NM-1 inhibiting and rhNMES1 promoting proliferation were confirmed in additional breast cancer cells. (FIG. 7A) Py8119 cells. (FIG. 7B) MCF-7 cells. Cells were labeled with luciferase, harvested 5 days after seeded and quantified by luciferase readout. Fold were calculated as d5 readout normalized by d0 luciferase readout (n=5 or 6). **P<0.01, ****P<0.0001; error bars indicate SD.

FIG. 8: Data from FIG. 5D shown in different scales to illustrate week 2 and week 3 data.

FIG. 9A-9B: mAb NM-1 inhibits proliferation in renal cell carcinoma cells. (FIG. 9A) DU145 cells. (FIG. 9B) UMCR6 cells. Cells were labeled with luciferase, harvested at the indicated days after seeded, quantified by luciferase readout. Fold were calculated by normalizing d0 luciferase readout (n=6). **P<0.01, ****P<0.0001; error bars indicate SD.

FIG. 10: Kinetic binding curves of anti-NMES1 antibodies measured using Octet instrument.

### DESCRIPTION OF ILLUSTRATIVE EMBODIMENTS

The present studies found a novel secreted micropeptide NMES1 (Normal Mucosa of Esophagus Specific 1, 83aa, also known as C15orf48) to be highly expressed in human breast cancer and further elevated in bone metastatic human cancer cells. In experimental mouse models, bone metastasis was increased by NMES1 overexpression but decreased by NMES1 knockdown in breast cancer cells. As a secreted protein, NMES1 can be detected in circulation, potentially representing a convenient biomarker for the prognosis of breast cancer malignancy as well as an accessible cancer drug target. Co-immunoprecipitation followed by proteomic analyses has identified transferrin receptor 1 (TFRC) as an NMES1 target. Mechanistic studies reveal that NMES1 binding to TFRC attenuates TFRC lysosomal degradation to enhance cancer cell iron uptake. Functional analyses show that TFRC deletion and NMES1 deletion in cancer cells exert similar effects on tumor growth and bone metastasis. Rescue experiments indicate that TFRC is an essential NMES1 target that is required for NMES1 regulation. Finally, pharmacological treatment with either an experimental polyclonal NMES1 antibody or a fully humanized monoclonal NMES1 antibody significantly reduced metastatic bone lesions, highlighting the exciting therapeutic potential of NMES1 blockade for the effective intervention of breast cancer bone metastasis.

Accordingly, in certain embodiments, the present disclosure provides methods for detecting secreted NMES1 micropeptide in a sample, such as for the detection or diagnosis of breast cancer, particularly breast cancer metastasis. Further provided herein are monoclonal antibodies to NMES1 which may be used for the treatment of cancer, such as breast cancer. Additional embodiments provide methods for treating cancer by targeting NMES1, such as by monoclonal antibodies provided herein, siRNA, or small molecule inhibitors of NMES1.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The term “about” means in general, the stated value plus or minus 5%.

“Treatment” and “treating” refer to administration or application of a therapeutic agent to a subject or performance of a procedure or modality on a subject for the purpose of obtaining a therapeutic benefit of a disease or health-related condition.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The terms “contacted” and “exposed,” when applied to a cell, are used herein to describe the process by which a construct and a therapeutic agent are delivered to a cell or are placed in direct juxtaposition with the target cell.

### II. NMES1 Antibodies

The NMES1 antibody may comprise an antibody or a fragment thereof that binds to at least a portion of NMES protein and inhibits NMES1 signaling. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Preferably, the NMES1 antibody is a monoclonal antibody or a humanized antibody.

In some embodiments, the NMES1 scFv comprises CDRs 1-3 of the heavy chain sequences in Table 2 or Table 4 and the CDRs 1-3 of the light chain sequences in Table 1 or Table 3. The NMES1 scFv may have at least 80%, such as 85%, 90%, 91%, 92%, 93%, 94%, 95%, 96%, 97%, 98%, 99%, or 100%, sequence identity to SEQ ID NOs:89-116.

TABLE 1
CDRs of light chain variable DNA
sequences of NMES1 antibodies.

| | Light Chains (LC) | | | | | |
|---|---|---|---|---|---|---|
| | | SEQ | | SEQ | | SEQ |
| Antibody | | ID | | ID | | ID |
| Name | CDR1 | NO | CDR2 | NO | CDR3 | NO |
| NMES1-1 | caaagcct | 1 | aaggt | 2 | atgcaaggt | 3 |
| | cgtacaca | | ttct | | gcacactgg | |
| | gtgatgga | | | | cctccgact | |
| | aacaccta | | | | | |
| | c | | | | | |
| NMES1-3 | cagagcat | 4 | tctgc | 5 | caacagagt | 6 |
| | tggcagct | | atcc | | tacagtggc | |
| | at | | | | ccgtacact | |
| NMES1-4 | cagggcat | 7 | gatgc | 8 | caacaggct | 9 |
| | tgccagtg | | ctcc | | cacagtacc | |
| | ct | | | | cgctcact | |
| NMES1-5 | agcagtga | 10 | gatgt | 11 | agctcatat | 12 |
| | cgttggtg | | cagt | | acaaggagc | |
| | catataac | | | | accactgtg | |
| | tat | | | | gta | |
| NMES1-6 | agcagtga | 13 | gaggt | 14 | agctcatat | 15 |
| | cgttggtg | | cagt | | gcaggcagc | |
| | gttataac | | | | aacaatttg | |
| | tat | | | | gta | |
| NMES1-7 | caggacat | 16 | acagc | 17 | ctacaagat | 18 |
| | cagaaatg | | aagt | | tacaagtat | |
| | at | | | | cctctcact | |
| NMES1-10 | agcagtga | 19 | gatgt | 20 | agctcatat | 21 |
| | cgttggtg | | cagt | | acaagcagc | |
| | gttataac | | | | agcactctc | |
| | tat | | | | gtggta | |

TABLE 2
CDRs of heavy chain variable DNA

| | Heavy Chains (HC) | | | | | |
|---|---|---|---|---|---|---|
| | | SEQ | | SEQ | | SEQ |
| Antibody | | ID | | ID | | ID |
| Name | CDR1 | NO | CDR2 | NO | CDR3 | NO |
| NMES | ggattca | 22 | atatcat | 23 | gcgagatt | 24 |
| 1-1 | ccttcag | | atgatgg | | gtcgaggt | |
| | tagctat | | aagtaat | | tcggggat | |
| | gct | | aaa | | gaaagcct | |
| | | | | | aaaagacc | |
| | | | | | ctgactac | |
| NMES | ggattca | 25 | attaatt | 26 | gcgagagc | 27 |
| 1-3 | cctttga | | ggaatgg | | ctctatgg | |
| | tgattat | | tggtagc | | ttcgggga | |
| | ggc | | aca | | gtgtatgc | |
| | | | | | ttttgata | |
| | | | | | tc | |
| NMES | ggaggca | 28 | atcatcc | 29 | gcgagagt | 30 |
| 1-4 | ccttcag | | ctatctt | | ttctgagc | |
| | caccta | | tggtaca | | tcggtgac | |
| | tgct | | aca | | tactacgc | |
| | | | | | tatggacg | |
| | | | | | tc | |
| NMES | ggttaca | 31 | atcagcg | 32 | gcgagagc | 33 |
| 1-5 | cctttac | | cttacaa | | aacaaggg | |
| | cagctat | | tggtaac | | gaaatgct | |
| | ggt | | aca | | tttgatat | |
| | | | | | c | |
| NMES | ggttaca | 34 | atcagcg | 35 | gcgagagc | 36 |
| 1-6 | cctttac | | cttacaa | | cgtagggc | |
| | cagctac | | tggtaac | | agctggcc | |
| | | | aca | | tttgacta | |
| | | | | | c | |
| NMES | ggctaca | 37 | atcagcg | 38 | gcgagagt | 39 |
| 1-7 | cctttaa | | gttacaa | | cggcctac | |
| | cagagat | | tggtaac | | agccggat | |
| | gct | | aca | | acaactat | |
| | | | | | ggaccac | |
| NMES | ggttaca | 40 | atcagcg | 41 | gcgagagc | 42 |
| 1-10 | cctttac | | cttacaa | | cacgttcg | |
| | cagctac | | tggtaac | | ggaatgct | |
| | ggt | | aca | | tttgatat | |
| | | | | | c | |

TABLE 3
CDRs of light chain amino acid
variable sequences of NMES1 antibodies

| | Light Chains (LC) | | | | | |
|---|---|---|---|---|---|---|
| | | SEQ | | SEQ | | SEQ |
| Antibody | | ID | | ID | | ID |
| Name | CDR1 | NO | CDR2 | NO | CDR3 | NO |
| NMES1-1 | QSLVHSDGNTY | 43 | KVS | 44 | MQGAHWPPT | 45 |
| NMES1-3 | QSIGSY | 46 | SAS | 47 | QQSYSGPYT | 48 |
| NMES1-4 | QGIAS A | 49 | DAS | 50 | QQAHSFPLT | 51 |
| NMES1-5 | SSDVGAYNY | 52 | DVS | 53 | SSYTRSTTVV | 54 |
| NMES1-6 | SSDVGGYNY | 55 | EVS | 56 | SSYAGSNNLV | 57 |
| NMES1-7 | QDIRND | 58 | TAS | 59 | LQDYKYPLT | 60 |
| NMES1-10 | SSDVGGYNY | 61 | DVS | 62 | SSYTSSSTLVV | 63 |

TABLE 4
CDRs of heavy chain amino acid
variable sequences of NMES1 antibodies

| | Heavy Chains (HC) | | | | | |
|---|---|---|---|---|---|---|
| | | SEQ | | SEQ | | SEQ |
| Antibody | | ID | | ID | | ID |
| Name | CDR1 | NO | CDR2 | NO | CDR3 | NO |
| NMES1-1_ | GFTFSSYA | 64 | ISYDGSNK | 65 | ARLSRFGD | 66 |
| | | | | | ESLKDPDY | |
| NMES1-3 | GFTFDDYG | 67 | INWNGGST | 68 | ARASMVRG | 69 |
| | | | | | VYAFDI | |
| NMES1-4 | GGTFSTYA | 70 | IIPIFGTT | 71 | ARVSELG | 72 |
| | | | | | DYYAMDV | |
| NMES1-5 | GYTFTSYG | 73 | ISAYNGNT | 74 | ARATRGN | 75 |
| | | | | | AFDI | |
| NMES1-6 | GYTFTSYG | 76 | ISAYNGNT | 77 | ARAVGQL | 78 |
| | | | | | AFDY | |
| NMES1-7 | GYTFNRDA | 79 | ISGYNGNT | 80 | ARVGLQP | 81 |
| | | | | | DTTMDH | |
| NMES1-10 | GYTFTSYG | 82 | ISAYNGNT | 83 | ARATFGN | 84 |
| | | | | | AFDI | |

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds NMES1.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against NMES1, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

### III. Treatment of Diseases

Further provided herein are methods for treating or delaying progression of cancer in an individual comprising administering an effective amount of a NMES1 therapy, such as a NMES antibody or siRNA.

A “therapeutic agent” as used herein refers to any agent that can be administered to a subject for the purpose of obtaining a therapeutic benefit of a disease or health-related condition. For example, nanoparticles that include a therapeutic agent may be administered to a subject for the purpose of reducing the size of a tumor, reducing or inhibiting local invasiveness of a tumor, or reducing the risk of development of metastases.

The therapeutic agent may be a small molecule, a peptide, a protein, a polypeptide, an antibody, an antibody fragment, a DNA, or an RNA. In particular embodiments, the therapeutic agent may be inhibitory RNA (RNAi), such as siRNA, shRNA, plasmid, mRNA, miRNA, or ncRNA, particularly siRNA or miRNA therapeutics. The miRNA may be a miRNA mimic, or a miRNA precursor. In particular embodiments, the therapeutic agent is a siRNA.

The term “siRNA” (short interfering RNA) refers to short double stranded RNA complex, typically 19-28 base pairs in length. In other words, siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). The complex often includes a 3′-overhang. siRNA can be made using techniques known to one skilled in the art and a wide variety of siRNA is commercially available from suppliers such as Integrated DNA Technologies, Inc. (Coralville, Iowa).

The size of the RNAi loaded used herein may be less than 100 nucleotides in length, such as less than 75 nucleotides, particularly less than 50 nucleotides in length. For example, the RNA may have a length of about 10-100 nucleotides, such as 20-50 nucleotides, particularly 10-20, 15-25, 20-30, 25-35, 30-40, or 45-50 nucleotides.

The RNAi may be modified or non-modified. The RNAi may comprise an alteration of one or more nucleotides. Such alterations can include the addition of non-nucleotide material, such as to the end(s) of the RNAi or internally (at one or more nucleotides of the RNA). In certain aspects, the RNAi molecule contains a 3′-hydroxyl group. Nucleotides in the RNAi molecules of the present disclosure can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation). Collectively, all such altered nucleic acids or RNAs described above are referred to as modified siRNAs. The RNAi may be conjugated or encapsulated for delivery, such as to lipids or nanoparticles.

Preferably, RNAi is capable of decreasing the expression of a protein by at least 10%, 20%, 30%, or 40%, more preferably by at least 50%, 60%, or 70%, and even more preferably by at least 75%, 80%, 90%, 95% or more.

The siRNA as used in the methods or compositions described herein may comprise a portion which is complementary to an mRNA sequence encoded by NCBI Reference Sequence for NMES1 (e.g., NCBI Reference Sequence: NM_032413.4). In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. In an embodiment, the overhang is UU. The siRNA can be 5′ phosphorylated or not and may be modified with any of the known modifications in the art to improve efficacy and/or resistance to nuclease degradation. In a non-limiting embodiment, the siRNA can be administered such that it is transfected into one or more cells. In one embodiment, a siRNA may comprise a double-stranded RNA comprising a first and second strand, wherein one strand of the RNA is 80, 85, 90, 95 or 100% complementary to a portion of an RNA transcript of a gene.

In one embodiment, a single strand component of a siRNA of the present disclosure is from 14 to 50 nucleotides in length. In another embodiment, a single strand component of a siRNA is 14, 15, 16, 17, 18, 19, 20, 21, 22, 23, 24, 25, 26, 27, or 28 nucleotides in length. In yet another embodiment, a single strand component of a siRNA of the present disclosure is 21 nucleotides in length. In yet another embodiment, a single strand component of a siRNA of the present disclosure is 22 nucleotides in length. In yet another embodiment, a single strand component of a siRNA of the present disclosure is 23 nucleotides in length. In one embodiment, a siRNA of the present disclosure is from 28 to 56 nucleotides in length.

Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and/or hairy cell leukemia.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

Therapeutically effective amounts of the compound can be administered by a number of routes, including parenteral administration, for example, intravenous, intraperitoneal, intramuscular, intrasternal, or intraarticular injection, or infusion. The therapeutically effective amount of the compound is that amount that achieves a desired effect in a subject being treated. For instance, this can be the amount of the compound necessary to inhibit advancement, or to cause regression of viral disease, or which is capable of relieving symptoms caused by viral disease.

The compound can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of the compound will be dependent on the subject being treated, the severity and type of the affliction, and the manner of administration. The exact amount of the compound is readily determined by one of skill in the art based on the age, sex, and physiological condition of the subject. Effective doses can be extrapolated from dose-response curves derived from in vitro or animal model test systems.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 μg/kg/body weight to about 1000 mg/kg/body weight (this such range includes intervening doses) or more per administration, and any range derivable therein. In non-limiting examples of a derivable range from the numbers listed herein, a range of about 5 μg/kg/body weight to about 100 mg/kg/body weight, about 5 μg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In certain embodiments, the compositions and methods of the present embodiments involve an albumin drug in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

The albumin drug conjugate may be administered before, during, after, or in various combinations relative to an additional cancer therapy, such as immune checkpoint therapy. The administrations may be in intervals ranging from concurrently to minutes to days to weeks. In embodiments where the albumin drug conjugate is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below anti-NMES1 therapy is “A” and an additional anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein transferase inhibitors, transplatinum, and pharmaceutically acceptable salts, acids, or derivatives of any of the above.

2. Immunotherapy

Various immunotherapies are known that may be used, including, e.g., anti-PD1 antibodies or compounds, anti-PD-L1 antibodies or compounds, anti-CTLA-4 antibodies or compounds, OX40 agonists, IDO inhibitors, anti-GITR antibodies or compounds, anti-LAGS antibodies or compounds, anti-TIM3 antibodies or compounds, anti-TIGIT antibodies or compounds, and anti-MERTK antibodies or compounds, an oncolytic virus immunotherapy, intratumoral injections; immunotherapies targeting STING, NLRP3, TLR9, CPG, TLR4, LTR7/8, OX40, or MER-tk; an anti-CTLA-4, anti-PD1, anti-PDL1, or anti-CD40 immunotherapy; FLT-3-ligand immunotherapies, and/or IL-2 cytokine immunotherapies. Additionally, the albumin drug conjugate could be combined with cell therapies, such as T cells, NK cells, or dendritic cells that may be engineered to express a CAR or TCR.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, Infection Immun., 66(11):5329-5336, 1998; Christodoulides et al., Microbiology, 144(Pt 11):3027-3037, 1998); cytokine therapy, e.g., interferons α, β, and γ, IL-1, GM-CSF, and TNF (Bukowski et al., Clinical Cancer Res., 4(10):2337-2347, 1998; Davidson et al., J. Immunother., 21(5):389-398, 1998; Hellstrand et al., Acta Oncologica, 37(4):347-353, 1998); gene therapy, e.g., TNF, IL-1, IL-2, and p53 (Qin et al., Proc. Natl. Acad. Sci. USA, 95(24):14411-14416, 1998; Austin-Ward and Villaseca, Revista Medica de Chile, 126(7):838-845, 1998; U.S. Pat. Nos. 5,830,880 and 5,846,945); and monoclonal antibodies, e.g., anti-CD20, anti-ganglioside GM2, and anti-p185 (Hanibuchi et al., Int. J. Cancer, 78(4):480-485, 1998; U.S. Pat. No. 5,824,311).

In some embodiments, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering. Isolation and transfer of tumor specific T cells has been shown to be successful in treating melanoma. Novel specificities in T cells have been successfully generated through the genetic transfer of transgenic T cell receptors or chimeric antigen receptors (CARs). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors.

In one embodiment, the present application provides for a combination therapy for the treatment of cancer wherein the combination therapy comprises adoptive T cell therapy. In one aspect, the adoptive T cell therapy comprises autologous and/or allogenic T cells. In another aspect, the autologous and/or allogenic T cells are targeted against tumor antigens.

3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

4. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—NMES1 Characterization

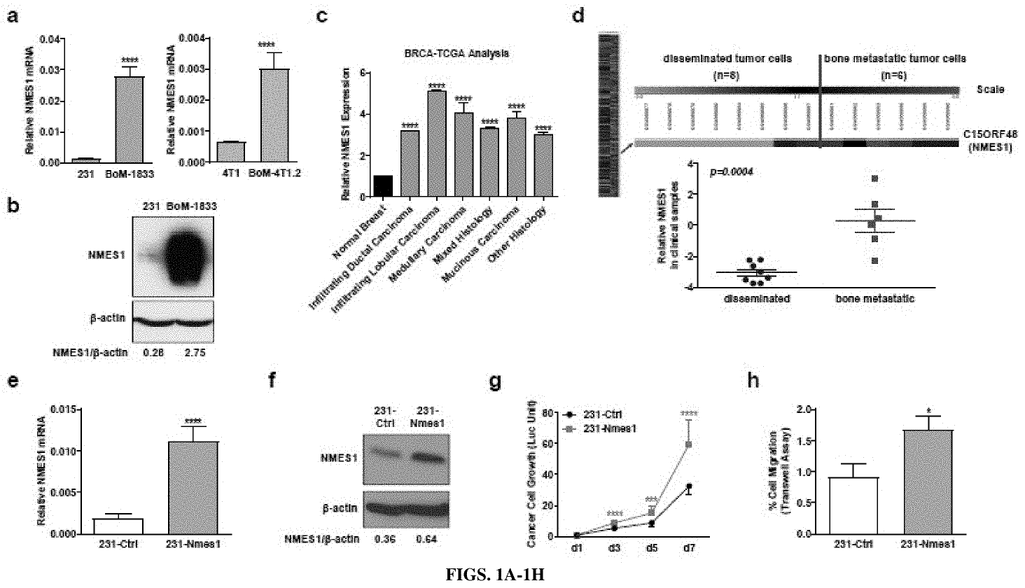

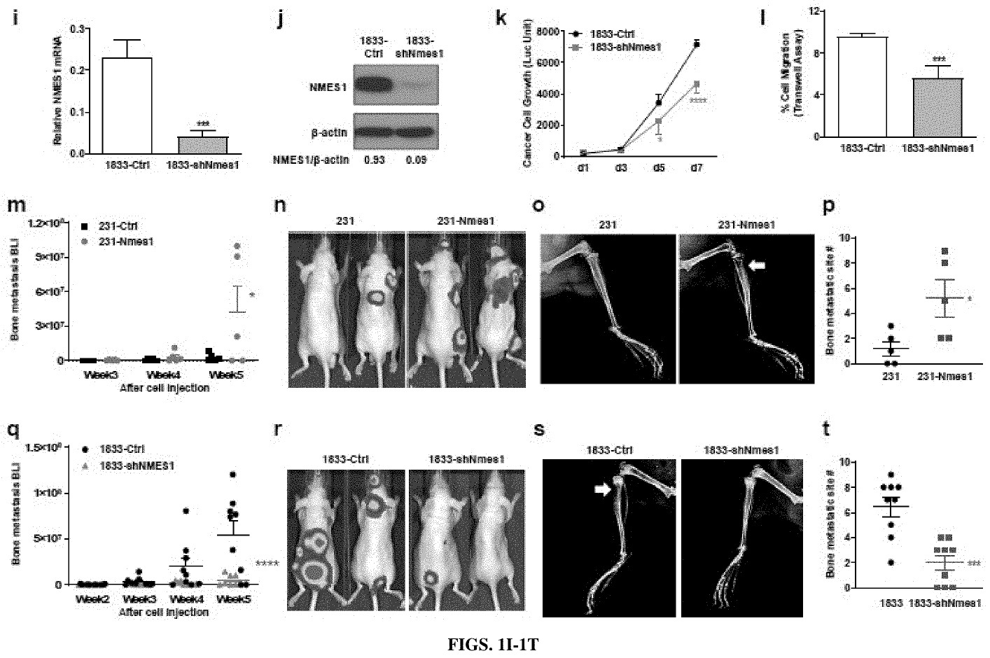

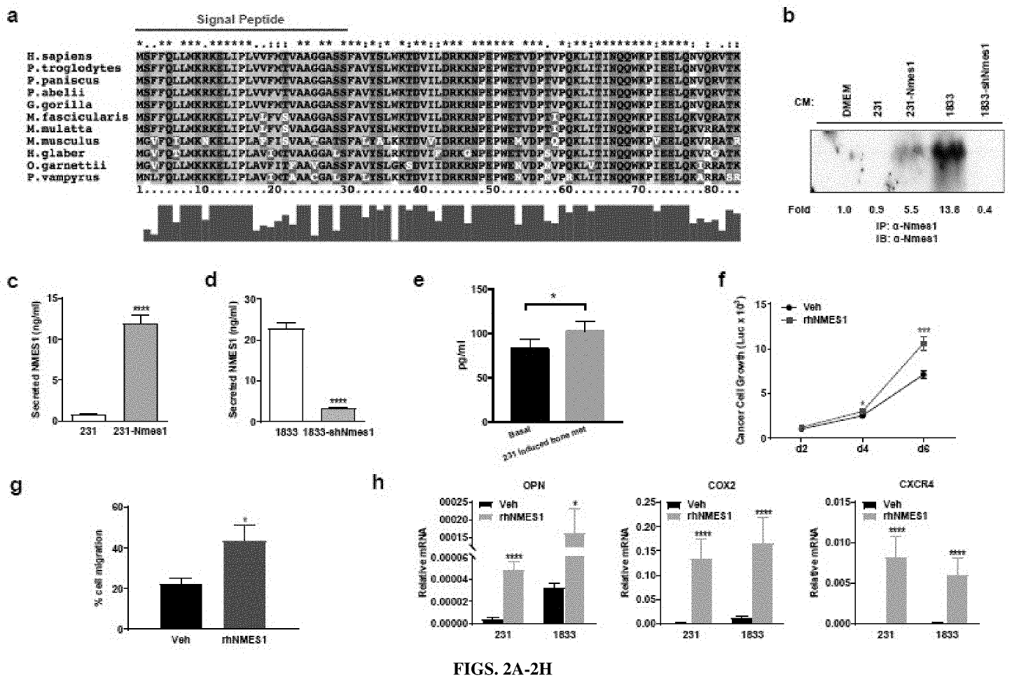

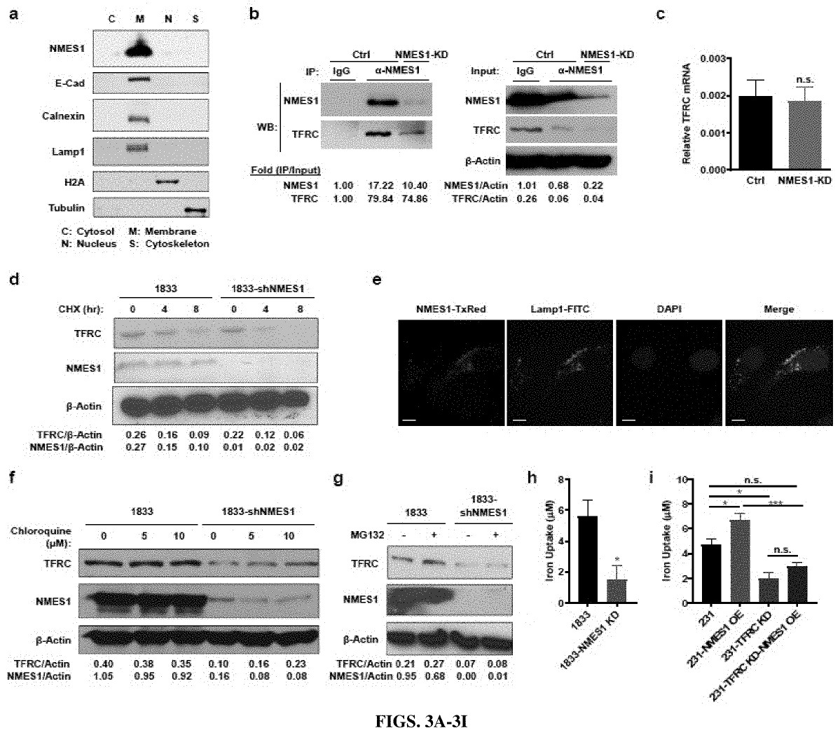

To identify genes that may exacerbate bone metastasis (met) of breast cancer, the RNA expression profiles of bone-met-prone breast cancer sublines and their parental lines, such as 1833 bone-met human breast cancer subline vs. MDA-MB-231 parental line, and 4T1.2 bone-met mouse mammary tumor subline vs. 4T1 parental line were compared. A group of genes were surveyed that showed elevated expression in breast cancers compared to normal tissue in databases. The results uncovered that the RNA expression of a functionally unknown micropeptide NMES1 (Normal Mucosa of Esophagus Specific 1, 83aa, 10 kDa), also known as C15orf48, was highly enriched in 1833 and 4T1.2 cells compared with MDA-MB-231 and 4T1 parental cells, respectively (FIG. 1A). Western blot showed that NMES1 protein was also more abundant in bone-met-prone subline (FIG. 1B). These findings suggest that NMES1 may promote bone metastasis of breast cancer.

To further determine the clinical significance of NMES1 in breast cancer and bone metastasis, bioinformatics analyses of human patient data was performed. First, the RNA-Seq and clinical data of breast invasive carcinoma (BRCA) from The Cancer Genome Atlas (TCGA) data portal was analyzed (Cancer Genome Atlas, 2012). The results revealed that NMES1 mRNA expression was significantly upregulated in all breast cancer types compared with normal breast tissue samples (FIG. 1C). This indicates that NMES1 may promote breast cancer progression. Second, a microarray dataset was analyzed that compared disseminated tumor cells obtained from bone marrow aspirates of breast cancer patients (controls) with metastatic tumor cells isolated from Computed Tomography guided biopsies of bone metastases (mets) (Cawthorn et al., 2009). The results showed that NMES1 expression was significantly higher in bone metastatic breast cancer cells than in disseminated breast cancer cells (FIG. 1D). This indicates that NMES1 may also exacerbate breast cancer metastasis to bone. These findings in human patients suggest that NMES1 regulation of breast cancer and bone metastasis may be evolutionally conserved.

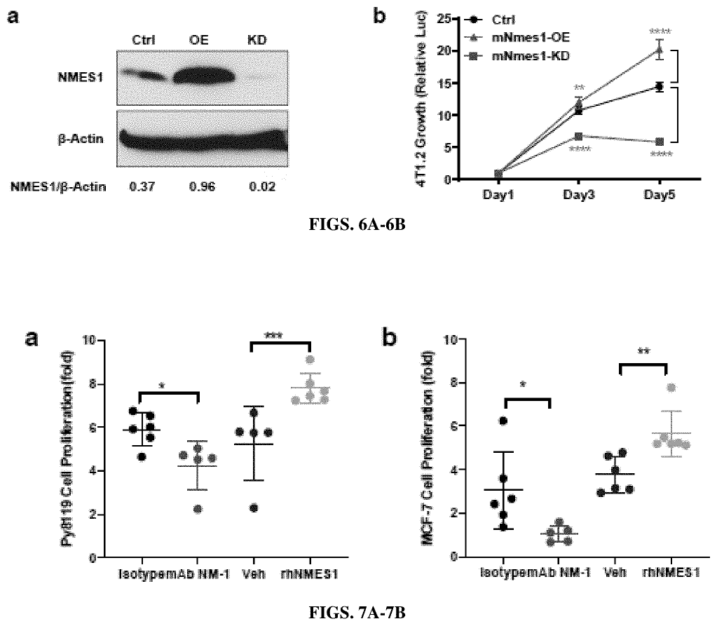

To determine if NMES1 overexpression (OE) in breast cancer cells enhances proliferation and migration, the MDA-MB-231 parental human breast cancer cell line was transduced with NMES1-expressing lentivirus or control lentivirus (Dharmacon) to derive 231-Nmes1 and 231-Ctrl stable clones. NMES1 RNA and protein expression was significantly elevated in 231-Nmes1 cells compared to 231-Ctrl cells (FIGS. 1E, F). This resulted in increased tumor cell proliferation (FIG. 1G) and migration (FIG. 1H). To determine if NMES1 knockdown (KD) in bone-met-prone human breast cancer cells suppresses proliferation and migration, BoM-1833 cells were transduced with NMES1-shRNAmir lentivirus or control-shRNAmir lentivirus (Dharmacon) to derive 1833-shNmes1 and 1833-Ctrl stable clones. NMES1 RNA and protein expression was significantly depleted in 1833-shNmes1 cells compared to 1833-Ctrl cells (FIG. 1I, J). This led to decreased tumor cell proliferation (FIG. 1K) and migration (FIG. 1L). Similar results were observed in experiments conducted in 4T1 and 4T1.2 cells (FIG. 6). These in vitro findings indicate that NMES1 may play a pro-tumor and pro-metastasis role in breast cancer cells.

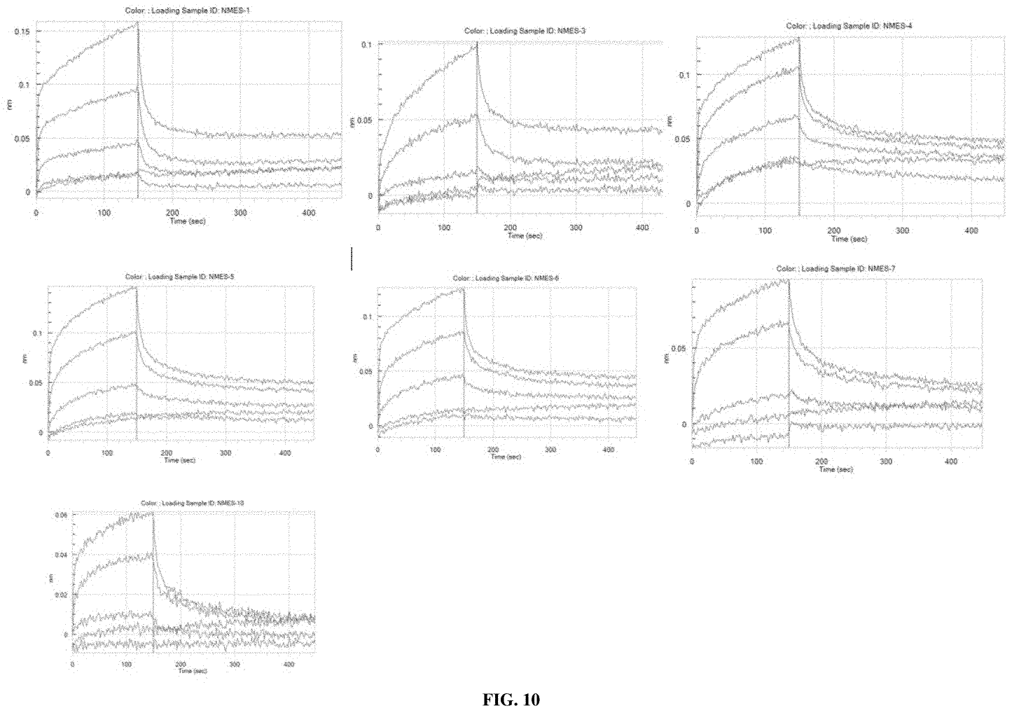

To determine if NMES1 OE or KD in human breast cancer cells impact bone metastasis in vivo, ultrasound-assisted intracardiac injection of luciferase-labelled cancer cells into female nu/nu mice was performed as previously described (Krzeszinski et al., 2014). Bone metastasis was quantified by weekly bioluminescence imaging (BLI) for both BLI signal intensity and bone met sites. Osteolytic lesion was assessed by high resolution X-ray imaging. The results showed that 231-Nmes1 cells develop significantly more bone metastasis than 231-Ctrl cells, demonstrated by the higher BLI signals (FIG. 1M-N), increased osteolytic lesions (FIG. 10) and larger number of metastatic sites in bone (FIG. 1P). In line with this observation, it was found that 1833-shNmes1 cells develop significantly less bone metastasis than 1833-Ctrl cells (FIG. 1Q-T). These in vivo findings further support a pro-bone-met role of NMES1 in breast cancer cells, and reveal NMES1 blockade as a potential strategy to impede metastatic bone diseases.

Bioinformatic analysis using YLoc, a web server for predicting subcellular localization, revealed that NMES1 is located in extracellular region with 99.9% confidence, harboring a highly conserved N-terminal signal peptide for protein secretion (FIG. 2A). Indeed, NMES1 protein was successfully detected in the culture medium of breast cancer cells using both immunoprecipitation (FIG. 2B) and ELISA (FIG. 2C-D). In accordance to NMES1 protein expression in the total cell lysate, the amount of secreted NMES1 in the culture medium was much higher in BoM-1833 cells compared with MDA-MB-231 cells, which was enhanced by NMES1 OE and reduced by NMES1 KD (FIG. 2B-D). Moreover, ELISA also detected NMES1 protein in mouse serum, the level of which was elevated upon the development of experimental bone metastasis (FIG. 2E). These data show that NMES1 is a micropeptide secreted from cancer cells into circulation, which may serve as a convenient cancer drug target.

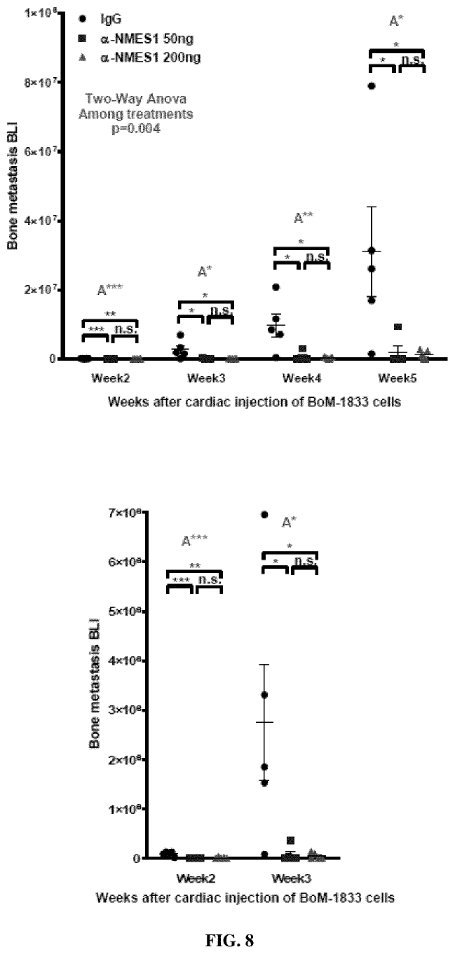

To examine the function of the secreted NMES1, recombinant human NMES1 (rhNMES1) protein was purified from insect cells. Acute treatment with rhNMES1 mimicked the effects of genetic NMES1 OE to increase 231 cancer cell growth (FIG. 2F) and migration (FIG. 2G). This effect extends to several other breast cancer cell lines including Py8119 and MCF-7 (FIG. 7). Moreover, rhNMES1 treatment elevated the expression of previously reported bone metastatic genes, including osteopontin (OPN), cyclooxygenase 2 (COX2) and C-X-C chemokine receptor type 4 (CXCR4) (FIG. 2H).

In line with the finding that NMES1 is a secreted protein, cell fractionation experiments showed that NMES1 was associated with the membrane fraction but absent in cytosol, nucleus or cytoskeleton, using Qproteome Cell Compartment Kit (Qiagen) (FIG. 3A). Because the membrane fraction contains not only plasma membranes but also intact organelles such as endosome, lysosome and the vesicles in the secretory pathway, NMES1 appearance in the membrane fraction supports it being a secreted peptide, yet this result also indicates NMES1 interaction with membrane receptors.

The working hypothesis is that, as a secreted protein, NMES1 functions mainly by interacting with membrane proteins and modulating their activity. To identify NMES1-binding targets, co-immunoprecipitation (co-IP) was performed followed by proteomics using both whole cell and membrane protein extract, with an isotype IgG as a negative control. UTSW Proteomic Core provided the technical expertise for mass spectrometry and bioinformatics analyses. Our results revealed that a top hit for NMES1-binding membrane proteins was transferrin receptor 1 (TFRC), which promotes breast cancer malignancy by enhancing cellular iron uptake.

Co-IP experiments showed that anti-NMES1 but not IgG control can pull down TFRC protein from control cancer cells but not NMES1-KD cancer cells (FIG. 3B), confirming the physical interaction between NMES1 and TFRC. Interestingly, it was found that NMES1 KD in cancer cells decreased TFRC protein levels without affecting TFRC mRNA expression (FIG. 3B-C). This was likely due to an increase in TFRC protein degradation rather than a reduction of TFRC protein synthesis because cycloheximide treatment did not abolish NMES1 regulation (FIG. 3D). Immunofluorescence staining showed that NMES1 co-localized with a widely used lysosome marker Lamp1 (FIG. 3E). This result agrees with the observation that NMES1 resides exclusively in the membrane fraction including the lysosome (FIG. 3A). Moreover, previous findings show that TFRC protein stability is mainly controlled by endocytosis followed by lysosomal degradation. In line with this observation and previous findings that TFRC protein stability is mainly controlled by endocytosis followed by lysosomal degradation, it was found that treatment with a lysosome inhibitor chloroquine dose-dependently rescued the lower TFRC protein in NMES1-KD cells (FIG. 3F). In contrast, treatment with a proteasome inhibitor MG132 had no rescuing effect (FIG. 3G). This suggests that NMES1 binding to TFRC prevents TFRC lysosomal degradation to enhance TFRC function.

Since TFRC is required for iron import from transferrin, it was tested whether NMES1 enhances cellular iron uptake. Cancer cells were cultured in serum-free RPMI-1640 medium with or without iron-saturated-transferrin overnight, and then total cellular iron levels were measured using an Iron Assay Kit. Iron uptake was calculated by subtracting the iron level in the presence of transferrin by the iron level in the absence of transferrin. The results showed that iron uptake was significantly reduced by NMES1 KD (FIG. 3H) but elevated by NMES1 OE (FIG. 3I). TFRC KD not only reduced the iron uptake to a similar degree as NMES1 KD (FIG. 3H, I), but also largely abolished the positive effect of NMES1 OE on iron import. These functional data support that NMES1 enhances cellular iron uptake through stabilizing TFRC.

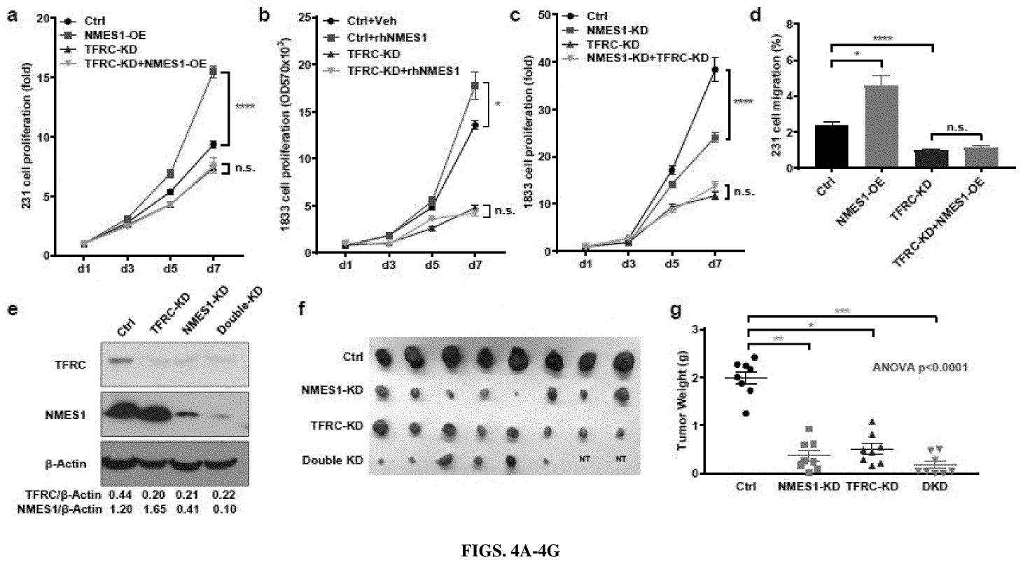

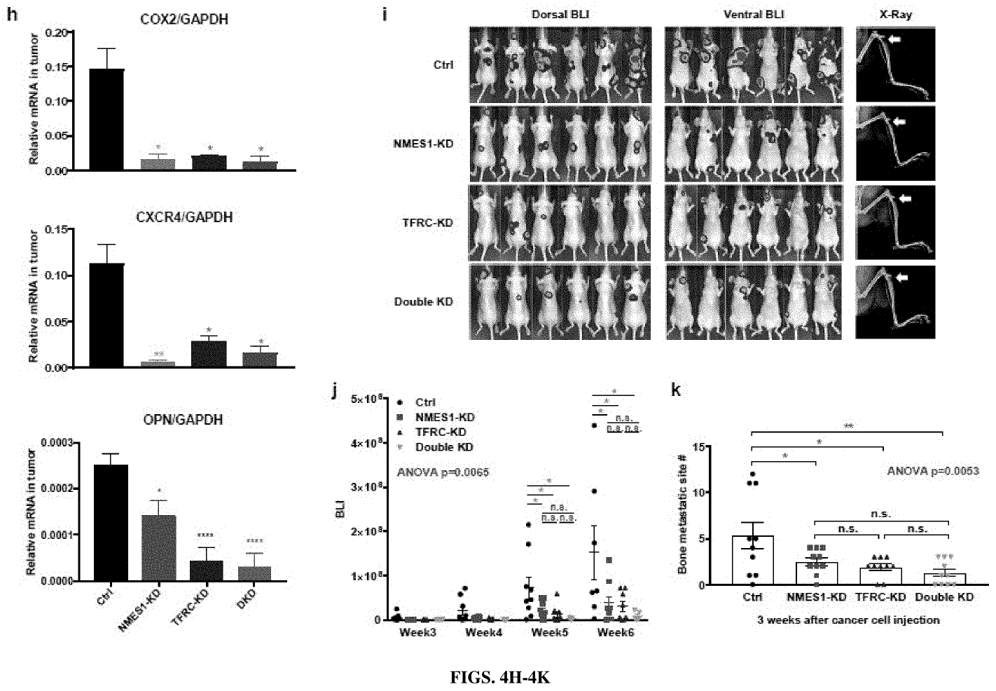

The role of TFRC was next examined in breast cancer cells and NMES1 regulation. TFRC KD not only reduced cancer cell proliferation and migration, but also completely abolished the effects of NMES1-OE, NMES1-KD or rhNMES1 treatment (FIG. 4A-D). The efficiency of TFRC-KD, NMES1-KD and double KD (DKD) was confirmed by western blot (FIG. 4E). Using an orthotopic mammary fat pad breast cancer cell transplantation model, it was found that tumor growth was diminished to a similar extent by NMES1-KD and TFRC-KD, and the absence of TFRC largely abolished the regulation by NMES1-KD (FIG. 4F, G). The expression of bone metastatic genes such as COX2 and CXCR4 and OPN was decreased by NMES1-KD, TFRC-KD and DKD (FIG. 4H). Using an intracardiac breast cancer cell transplantation model, it was found that bone metastasis was also blunted to a similar degree by NMES1-KD and TFRC-KD shown by the significantly lower BLI signal and less bone metastatic sites (FIG. 4I-K); moreover, the absence of TFRC largely abolished the regulation of bone metastasis by NMES1-KD (FIG. 4I-K). Together, these findings support that TFRC is a key NMES1 target that mediates the tumor-promoting and bone-met-enhancing functions of NMES1.

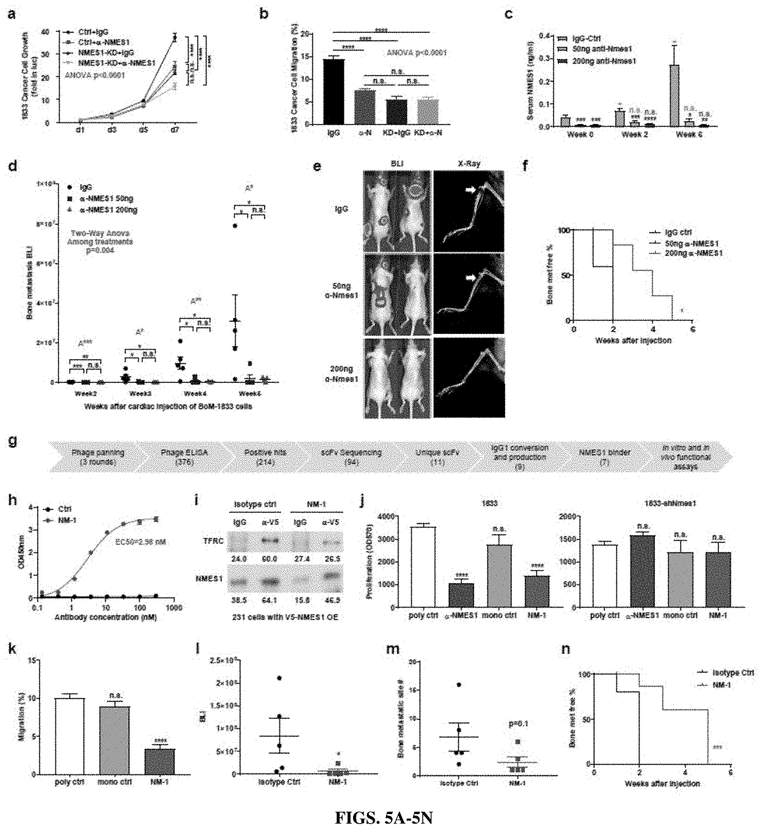

Since NMES1 is a secreted micropeptide in circulation, it was next explored whether NMES1 blocking antibody could serve as an effective therapeutic strategy to impede breast cancer bone metastasis. A commercial polyclonal anti-NMES1 (α-NMES1) antibody was tested first. In vitro assays showed that anti-NMES1 treatment significantly reduced breast cancer cell proliferation in 1833 cells but not in the NMES1-KD negative control cells (FIG. 5A, B). In vivo analyses showed that anti-NMES1 treatment decreased serum NMES1 levels in a dose-dependent manner (FIG. 5C), effectively blunted bone metastasis (FIG. 5D) and osteolytic lesion (FIG. 5E), leading to a higher bone-met-free survival (FIG. 5F).

Monoclonal antibodies (mAbs, named as NM-1 to NM-10) were further identified by panning a phage display human scFv antibody library using recombinantly produced human NMES1 protein (rhNMES1) (FIG. 5G-H). Co-IP analysis showed that monoclonal antibody NM-1 was able to reduce the interaction between NMES1 and TFRC (FIG. 5I). Functional studies identified four monoclonal antibodies (NM-1, 3, 4, 7) that could significantly inhibit the proliferation of 1833 cancer cells but not NMES1-KD negative control cells (FIG. 5J). These four mAbs also reduced cancer cell migration (FIG. 5K). Effect of mAb NM-1 on proliferation was also observed in additional breast cancer cells as well as renal cell carcinoma cells (FIGS. 7, 8). Further in vivo screening showed that out of the four candidates, only NM-1 effectively suppressed bone metastasis (FIG. 5L-Mp=0.1 for FIG. 5M), leading to a better bone-met-free survival (FIG. 5N). These results further support the anti-bone-met effects of NMES1 blockade and provide a new therapeutic agent for future clinical development.

### Example 2—Materials and Methods

Reagents: The bone-metastasis-prone MDA-MB-231 human breast cancer cell sub-line (MDA231-BoM-1833) was provided by Joan Massagué (a Howard Hughes Medical Institute Investigator at Memorial Sloan-Kettering Cancer Center). Py8119 mouse breast cancer cell line was provided by Lesley Ellies (UC San Diego). MCF-7 cell line was obtained from ATCC. RCC cell lines DU145 and UMCR6 were provided by James Brugarolas (UT Southwestern). All the cell lines were labeled with luciferase in the lab. Lentiviral vectors were purchased for human NMES1 expression clone (Thermo Fisher Scientific), human NMES1 shRNA (Open Biosystems) and human TFRC shRNA (Sigma). Cancer cell sublines with gene overexpression or knockdown were obtained by lentiviral transduction. Antibodies were purchased for NMES1 (sc-138479, Santa Cruz Biotechnologies) and TFRC (10F11, Thermo Fisher Scientific). NMES1 ELISA kit was purchased from MyBioSource.

Cancer cell analyses: To measure cancer cell proliferation, luciferase-labeled cancer cells were seeded in 96-well plates at a density of 10⁴ cells per well in Dulbecco's modified Eagle's medium (DMEM) containing 10% fetal bovine serum (FBS). Culture medium were replenished every 3 days and supplemented with either rhNMES1 protein or PBS control as indicated. Cells were lysed at the indicated time points. Luciferase output from the cell lysate was quantified to measure cancer cell numbers. Cancer cell migration was quantified using a transwell assay. Cells and chambers were prepared following the Cell Migration Protocol from Corning (Tewksbury, Mass.). Briefly, 5×10⁵ cells were seeded in the upper chamber and 600 μl culture medium+10% FBS were placed in the bottom chamber. After 48 hours, cells in upper and lower chambers were quantified by luciferase output, and percentage of cell migration was calculated. Primary tumor growth was measured by orthotopic injection of human breast cancer cells into the mammary fat pad of nude mice as previously described (Krzeszinkski et al., 2014). Briefly, 100 μl of 5×10⁵ cancer cell/MATRIGEL® mixture were injected into mouse fat pad #4 through a small incision. Five weeks after implantation, mice were sacrificed and tumor lumps were dissected out and weighed.

Bone metastasis analysis: Using a Visual Sonics Vevo770 small-animal ultrasound device, luciferase-labelled cancer cells were injected into the left cardiac ventricle at a dose of 10⁵ cells/100 μl PBS as previously described (Krzeszinkski et al., 2014). Bone metastases were detected and quantified weekly by BLI using a Caliper Xenogen Spectrum instrument at University of Texas Southwestern small animal imaging core facility. The osteolytic metastatic lesions were imaged by radiography using a Faxitron Cabinet X-ray System with the X-ray tube peak kilo-voltage fixed at 26 kVp and the exposure time at 15s. For in vivo treatment with polyclonal anti-NMES1, the mice were pretreated with 3 doses of antibody or IgG control during the week before cardiac injection of cancer cells and continuously treated three times a week at a low dose of 50 ng per mouse and a high dose of 200 ng per mouse. For in vivo treatment with humanized monoclonal anti-NMES1, all antibodies were injected at 2 μg per mouse, and the first treatment started right after the injection of cancer cells and continued three times a week.

Gene Expression Analyses: For mRNA expression, RNA was reverse transcribed into cDNA using an ABI High Capacity cDNA RT Kit (Life Technologies) and then analyzed using real-time quantitative PCR (SYBR Green) in triplicate. PCR primer sequences are shown below. All mRNA expression was normalized by L19.

hNMES1-F
(SEQ ID NO: 117)
AACTCATTCCCTTGGTGGTG
hNMES1-R
(SEQ ID NO: 118)
GTAGGGTCCACAGTTTCCCA
mNMES1-F
(SEQ ID NO: 119)
GGAAAAGAAACCCAGAGCCT
mNMES1-R
(SEQ ID NO: 120)
GGACTTTTTGCAGCTCCTCA
hTFRC-F
(SEQ ID NO: 121)
TGTGGGGAAGGGGCTGT
hTFRC-R
(SEQ ID NO: 122)
CCACCAAACAAGTTAGAGAATGC
hOPN-F
(SEQ ID NO: 123)
GTGATTTGCTTTTGCCTCCT
hOPN-R
(SEQ ID NO: 124)
GCCACAGCATCTGGGTATTT
hCOX-2-F
(SEQ ID NO: 125)
CCCTTCTGCCTGACACCTTTC
hCOX-2-R
(SEQ ID NO: 126)
GCAACCCTGCCAGCAATTT
hCXCR4-F
(SEQ ID NO: 127)
CGCTACCTGGCCATCGTC
hCXCR4-R
(SEQ ID NO: 128)
CATAGACCACCTTTTCAGCCAAC

Iron uptake assay: Total iron concentration was measured by iron assay kit from BioVison Inc according to the manufacture's instruction. Cellular iron level in cancer cells cultured in serum-free RPMI-1640 medium overnight (24 hours) were considered as base level. The surplus of total iron from cancer cells cultured in the same condition plus 5m/m1 iron-saturated transferrin were calculated as iron uptake.

Generation of monoclonal antibodies for human NMES1: A panel of 7 human monoclonal antibodies that bind to human NMES1 was selected by panning an in-house premade phage display library of human single-chain variable antibody fragments (scFv). This library was constructed starting with PBMC and tonsil samples from different donors. Total RNA isolated from these samples was used to synthesize cDNA. The library was then generated PCR amplification and assembly of VL and VH immunoglobulin domains with a linker followed by cloning into a phage vector. For phage panning, biotinylated recombinant NMES1 protein was used as the target for selection. In the first round of panning, 7.8×10¹² phage particles displaying scFv library were incubated with 5% milk and streptavidin beads (Dynabeads streptavidin T1, Invitrogen) to de-select phage particles with non-specific binding to streptavidin. Phage particles displaying scFv fragments without non-specific binding to streptavidin were then incubated with 1.5 μM biotinylated NMES1 bait. After washing four times with PBST, the phage particles, now enriched for antigen specificity, were pulled down using a magnet and eluted using Triethylamine (TEA). The eluates were used to infect E. coli TG1 cells. Cells were cultured for phage amplification. In subsequent rounds of selection, amplified phage particles were used as input. Similar panning procedures were used in the second and third rounds. Two exceptions were that the antigen concentrations were reduced (500 nM for the second round and 200 nM for the third round) and washing times were increased (6 times for the second round and 10 times for the third round). After three rounds of panning, the phage eluates were used to infect E. coli TG1 cells, which were subsequently plated for growth and selection of single colonies. A total of 376 single colonies were picked to amplify phage for ELISA to test for binding with NMES1. A total of 214 colonies were positive. Then, 94 of the 214 colonies were selected for sequencing and analysis of their scFv sequences. The 11 scFvs with unique amino acid sequences were converted to full IgG1 heavy chain and light chain constructs. These constructs were co-transfected into human embryonic kidney freestyle 293 (HEK293F) cells for production of recombinant antibodies according to manufacturer's instructions. Nine antibodies with reasonable yield were produced. Seven of these were confirmed to bind NMES1. These seven antibodies were subjected to in vitro functional screening. Four of these antibodies were subjected to in vivo functional test.

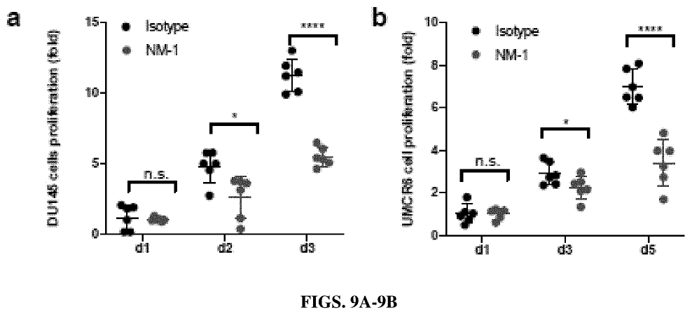

CDRs of the anti-NMES1 monoclonal antibodies are listed in Tables 1-4. Binding of NMES1 by monoclonal antibodies was first screened by ELISA using supernatants collected from expression cultures. ELISA concentration titration assay was used to determine the binding affinity of a panel of monoclonal antibodies to NMES1 antigen (FIG. 5H). Binding constants (EC50) of a panel of monoclonal antibodies were estimated by titration ELISA and data is analyzed using the 4 parameter curve fitting with GraphPad Prism program (Table 5). For antibody affinity measurement, antibody (30 μg/mL) was loaded onto the protein A biosensors for 4 min. Following a short baseline in kinetics buffer, the loaded biosensors were exposed to a series of recombinant NMES1 protein at 0-900 nM and background subtraction was used to correct for baseline drifting. All experiments were performed with shaking at 1,000 rpm. Background wavelength shifts were measured from reference biosensors that were loaded only with antibody with antigen. Kinetic sensorgrams for each of the antibodies are shown in FIG. 9. ForteBio's data analysis software was used to fit the data to a 1:1 binding model to extract an association rate and dissociation rate. The K_{D} was calculated using the ratio of k_{off}/kₒₙ and the estimated values of K_{D} for NMES1 mAbs in Table 6. Pairwise binding competition among anti-NMES1 mAbs was used to determine the binding epitopes of each mAbs using Octet instrument and protein A biosensors.

TABLE 5
EC₅₀ of anti-NMES1 monoclonal antibodies

| Mab Name | EC₅₀ (nM) |
|---|---|
| NMES-#1 | 2.98 |
| NMES-#3 | 40.6 |
| NMES-#4 | 4.37 |
| NMES-#5 | 0.216 |
| NMES-#6 | 2.39 |
| NMES-#7 | 14.38 |
| NMES-#10 | 305.7 |

TABLE 6
Kinetic binding constants determined using Octet
instrument (FIG. 7)

| Antibody Name | KD (M) | kon(l/Ms) | kdis(1/s) | R² |
|---|---|---|---|---|
| NMES-1 | 1.55E−08 | 3.23E+05 | 4.99E−03 | 0.6786 |
| NMES-3 | 5.60E−08 | 5.38E+04 | 3.01E−03 | 0.6756 |
| NMES-4 | 1.71E−08 | 2.23E+05 | 3.82E−03 | 0.737 |
| NMES-5 | 2.79E−08 | 1.66E+05 | 4.63E−03 | 0.7631 |
| NMES-6 | 1.95E−08 | 2.27E+05 | 4.41E−03 | 0.7525 |
| NMES-7 | 3.98E−08 | 1.50E+05 | 5.95E−03 | 0.8366 |
| NMES-10 | 8.99E−08 | 1.46E+05 | 1.31E−02 | 0.8885 |

ELISA EC₅₀ determination: Corning high binding assay plates were coated with recombinant NMES1 protein (1 μg/mL) at 4° C. overnight and blocked with 5% skim milk at 37° C. for 2 hours. Serially diluted anti-NMES1 antibodies were added at a volume of 100 μl per well for incubation at 37° C. for 2 hours. Anti-human IgG Fc HRP-conjugated antibody was diluted 1:5000 and added at a volume of 100 μl per well for incubation at 37° C. for 1 hour. The plates were washed 3 times with PBST (0.05% Tween-20) between incubation steps. TMB substrate was added 100 μl per well for color development and 2 M H2504 was added 50 μl per well to stop the reaction. The absorbance at 450 nm was read by SpectraMax microplate reader. For EC50 determination, data points were plotted by GraphPad Prism8. EC50 values were calculated using a three-parameter nonlinear model.

Statistical Analyses: Statistical analyses were performed with Student's t-Test and represented as mean±standard deviation (SD) or standard error of mean (SEM) as indicated in figure legend. For multi-group comparison, two-way ANOVA was used. No animal or sample was excluded from the analysis. The p values were designated as: *, p<0.05; **, p<0.01; ***, p<0.005; ****, p<0.001; n.s. non-significant (p>0.05).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Austin-Ward and Villaseca, Revista Medica de Chile, 126(7):838-845, 1998.
Ausubel et al., Current Protocols in Molecular Biology, Greene Publishing Associates and John Wiley & Sons, N Y, 1994.
Bukowski et al., Clinical Cancer Res., 4(10):2337-2347, 1998.
Cancer Genome Atlas. Nature 490, 61-70, 2012.
Cawthorn, et al. Clinical & experimental metastasis 26, 935-943, 2009.
Christodoulides et al., Microbiology, 144(Pt 11):3027-3037, 1998.
Coleman, Cancer Treat Rev 27, 165-176, 2001.
Coleman. Nature reviews. Clinical oncology 9, 76-78, 2012.
Davidson et al., J. Immunother., 21(5):389-398, 1998.
Hanibuchi et al., Int. J. Cancer, 78(4):480-485, 1998.
Hellstrand et al., Acta Oncologica, 37(4):347-353, 1998.
Hui and Hashimoto, Infection Immun., 66(11):5329-5336, 1998.
Krzeszinski, et al. Nature 512, 431-435, 2014.
Qin et al., Proc. Natl. Acad. Sci. USA, 95(24):14411-14416, 1998.
U.S. Pat. No. 5,739,169
U.S. Pat. No. 5,801,005
U.S. Pat. No. 5,824,311
U.S. Pat. No. 5,830,880
U.S. Pat. No. 5,846,945

1. An isolated monoclonal antibody, wherein the antibody specifically binds secreted normal mucosa of esophagus specific 1 (NMES1), wherein the antibody comprises:
(a) V_{L} domain CDRs 1-3 (SEQ ID NOs: 43-45) and V_{H} domain CDRs 1-3 (SEQ ID NOs:63-66);
(b) V_{L} domain CDRs 1-3 (SEQ ID NOs: 46-48) and V_{H} domain CDRs 1-3 (SEQ ID NOs: 67-69);
(c) V_{L} domain CDRs 1-3 (SEQ ID NOs: 49-51) and V_{H} domain CDRs 1-3 (SEQ ID NOs:70-72);
(d) V_{L} domain CDRs 1-3 (SEQ ID NOs: 52-54) and V_{H} domain CDRs 1-3 (SEQ ID NOs:73-75);
(e) V_{L} domain CDRs 1-3 (SEQ ID NOs: 55-57) and V_{H} domain CDRs 1-3 (SEQ ID NOs:76-78);
(f) V_{L} domain CDRs 1-3 (SEQ ID NOs: 58-60) and V_{H} domain CDRs 1-3 (SEQ ID NOs:79-81); or
(g) V_{L} domain CDRs 1-3 (SEQ ID NOs: 61-63) and V_{H} domain CDRs 1-3 (SEQ ID NOs:82-84).

2. The antibody of claim 1, wherein the antibody comprises V_{L} domain CDRs 1-3 (SEQ ID NOs: 43-45) and V_{H} domain CDRs 1-3 (SEQ ID NOs:63-66).

3. The antibody of claim 2, wherein the antibody comprises a V_{H} domain at least about 80% identical to the V_{H} domain (SEQ ID NO: 103) of NMES-1 and a V_{L} domain (SEQ ID NO:110) at least about 80% identical to the V_{L} domain of NMES-1.

4. The antibody of claim 3, wherein the antibody comprises a V_{H} domain identical to the V_{H} domain (SEQ ID NO: 103) of NMES-1 and a V_{L} domain (SEQ ID NO: 110) identical to the V_{L} domain NMES-1.

5. The antibody of claim 1, wherein the antibody comprises a V_{H} domain at least about 80% identical to the V_{H} domain (SEQ ID NOs:104-109) of NMES-3, NMES-4, NMES-5, NMES-6, NMES-7, or NMES-10 and a V_{L} domain (SEQ ID NOs: 111-116) at least about 80% identical to the V_{L} domain of NMES-3, NMES-4, NMES-5, NMES-6, NMES-7, or NMES-10.

6. The antibody of claim 3, wherein the antibody comprises a V_{H} domain identical to the V_{H} domain (SEQ ID NO: 104-109) of NMES-3, NMES-4, NMES-5, NMES-6, NMES-7, or NMES-10 NMES-1 and a V_{L} domain (SEQ ID NO: 111-116) identical to the V_{L} domain NMES-3, NMES-4, NMES-5, NMES-6, NMES-7, or NMES-10.

7. The antibody of claim 1, wherein the antibody is recombinant.

8. The antibody of claim 1, wherein the antibody is an IgG, IgM, IgA or an antigen binding fragment thereof.

9. The antibody of claim 1, wherein the antibody is a Fab′, a F(ab′)2, a F(ab′)3, a monovalent scFv, a bivalent scFv, or a single domain antibody.

10. The antibody of claim 1, wherein the antibody is a human, humanized antibody or de-immunized antibody.

11. The antibody of claim 1, wherein the antibody is conjugated to an imaging agent, a chemotherapeutic agent, a toxin or a radionuclide.

12. A composition comprising an antibody of claim 1 in a pharmaceutically acceptable carrier.

13. An isolated polynucleotide molecule comprising a nucleic acid sequence encoding an antibody of claim 1.

14. A recombinant polypeptide comprising an antibody V_{H} domain comprising: (a) V_{L} domain CDRs 1-3 (SEQ ID NOs: 43-45) and V_{H} domain CDRs 1-3 (SEQ ID NOs:63-66);
(b) V_{L} domain CDRs 1-3 (SEQ ID NOs: 46-48) and V_{H} domain CDRs 1-3 (SEQ ID NOs: 67-69);
(c) V_{L} domain CDRs 1-3 (SEQ ID NOs: 49-51) and V_{H} domain CDRs 1-3 (SEQ ID NOs:70-72);
(d) V_{L} domain CDRs 1-3 (SEQ ID NOs: 52-54) and V_{H} domain CDRs 1-3 (SEQ ID NOs:73-75);
(e) V_{L} domain CDRs 1-3 (SEQ ID NOs: 55-57) and V_{H} domain CDRs 1-3 (SEQ ID NOs:76-78);
(f) V_{L} domain CDRs 1-3 (SEQ ID NOs: 58-60) and V_{H} domain CDRs 1-3 (SEQ ID NOs:79-81); or
(g) V_{L} domain CDRs 1-3 (SEQ ID NOs: 61-63) and V_{H} domain CDRs 1-3 (SEQ ID NOs:82-84).

15. An isolated polynucleotide molecule comprising a nucleic acid sequence encoding a polypeptide of claim 14.

16. A host cell comprising one or more polynucleotide molecule(s) encoding an antibody of claim 1.

17. The host cell of claim 16, wherein the host cell is a mammalian cell, a yeast cell, a bacterial cell, a ciliate cell or an insect cell.

18.-20. (canceled)

21. A method for treating cancer in a subject comprising administering an effective amount of an inhibitor of NMES1 to the subject.

22. The method of claim 21, wherein the NMES1 inhibitor is an antibody of claim 1.

23.-35. (canceled)

36. A method for detecting a cancer in a subject comprising measuring elevated secreted NMES1 relative to a control in a sample from the subject, such as wherein the method is an in vitro method and the NMES1 is secreted.

37.-48. (canceled)
